SEQUENCE LISTING (19 sequences)

SEQ ID NO: 1 - DNA, 105 bp, synthetic construct
  source 1..105: mol_type = other DNA
aatcttatgtggcacagcccagtaagattgctatcatttaaattaatatatgaagttcctatactttctagagaataggaacttcgtgtaggctggagctgcttc

SEQ ID NO: 2 - DNA, 105 bp, synthetic construct
  source 1..105: mol_type = other DNA
aaaaacaagacagatttcaatttttcattaacaggttaagaggtaattaagaagttcctattctctagaaagtataggaacttcaatgggaattagccatggtcc

SEQ ID NO: 3 - DNA, 35 bp, synthetic construct
  source 1..35: mol_type = other DNA
cgtataacccataatcaattttatgacaagaatcc

SEQ ID NO: 4 - DNA, 29 bp, synthetic construct
  source 1..29: mol_type = other DNA
cacggaggtaaaattgataaacagttagc

SEQ ID NO: 5 - DNA, 20 bp, synthetic construct
  source 1..20: mol_type = other DNA
gcaatgcgggaaacagactc

SEQ ID NO: 6 - DNA, 20 bp, synthetic construct
  source 1..20: mol_type = other DNA
tttctctgctgtgccgaata

SEQ ID NO: 7 - DNA, 21 bp, synthetic construct
  source 1..21: mol_type = other DNA
gctgctaagtacctcctggtt

SEQ ID NO: 8 - DNA, 22 bp, synthetic construct
  source 1..22: mol_type = other DNA
ggtatctgttccgcttggactg

SEQ ID NO: 9 - DNA, 70 bp, synthetic construct
  source 1..70: mol_type = other DNA
gccatatcaatactggtgtagtaaccatcgacgcaacgaaaactgacgtagtgtaggctggagctgcttc

SEQ ID NO: 10 - DNA, 70 bp, synthetic construct
  source 1..70: mol_type = other DNA
atggtgttattggttccgcgtgcattgttatctatgatacggggtcactaatgggaattagccatggtcc

SEQ ID NO: 11 - DNA, 24 bp, synthetic construct
  source 1..24: mol_type = other DNA
cctctataacctgaacatgcttgg

SEQ ID NO: 12 - DNA, 21 bp, synthetic construct
  source 1..21: mol_type = other DNA
gatggcgctgctcttctattg

SEQ ID NO: 13 - DNA, 33 bp, synthetic construct
  source 1..33: mol_type = other DNA
gatcgagctcatgcatttcctttcttcctcttc

SEQ ID NO: 14 - DNA, 29 bp, synthetic construct
  source 1..29: mol_type = other DNA
gatcctcgagttctcctacttcaacgttc

SEQ ID NO: 15 - DNA, 23 bp, synthetic construct
  source 1..23: mol_type = other DNA
cctttctcgctacttagtccgtc

SEQ ID NO: 16 - DNA, 22 bp, synthetic construct
  source 1..22: mol_type = other DNA
ggtcctgagtttcagacaaggc

SEQ ID NO: 17 - DNA, 22 bp, synthetic construct
  source 1..22: mol_type = other DNA
ccacagaactcagagacaggtg

SEQ ID NO: 18 - DNA, 22 bp, synthetic construct
  source 1..22: mol_type = other DNA
cggcgatacttgatgccttctc

SEQ ID NO: 19 - DNA, 23 bp, synthetic construct
  source 1..23: mol_type = other DNA
caggaatgcatcggctgacangg

(19) United States
(12) Patent Application Publication
(10) Pub. No.: US 2026/0098073 A1
(43) Pub. Date: Apr. 9, 2026

(54) COMPOSITIONS AND METHODS FOR INHIBITION OF E. COLI HEMOLYSIN DURING URINARY TRACT INFECTION

(71) Applicants: David Hunstad, St. Louis, MO (US); Hunter Kuhn, St. Louis, MO (US); Michael Diamond, St. Louis, MO (US); Hongming Ma, St. Louis, MO (US)

(72) Inventors: David Hunstad, St. Louis, MO (US); Hunter Kuhn, St. Louis, MO (US); Michael Diamond, St. Louis, MO (US); Hongming Ma, St. Louis, MO (US)

(73) Assignee: Washington University, St. Louis, MO (US)

(21) Appl. No.: 19/349,990

(22) Filed: Oct. 4, 2025

Related U.S. Application Data
(60) Provisional application No. 63/703,341, filed on Oct. 4, 2024.

(51) Int. Cl. C07K 14/705; A61K 38/00; A61P 13/02

(52) U.S. Cl. CPC C07K 14/705; A61P 13/02; A61K 38/00; C07K 2319/30

(57) ABSTRACT

Compositions and methods for treating urinary tract infection (UTI) and/or sepsis in a subject in need thereof are provided. Methods include administering to the subject a composition comprising an alpha-hemolysin (HlyA) inhibiting agent. In some embodiments, the HlyA inhibiting agent includes a soluble low-density lipoprotein receptor (LDLR)-Fc fusion protein as described herein, a clathrin-mediated endocytosis (CME) inhibitor, and/or an anti-LDLR antibody. In some embodiments, the soluble LDLR-Fc fusion protein includes an Fc domain and at least one LDLR type A domain. In some embodiments, the UTI and/or sepsis is caused by E. coli.

### CROSS-REFERENCE TO RELATED APPLICATIONS

This application claims priority from U.S. Provisional Application Ser. No. 63/703,341 filed 4 Oct. 2024, which is incorporated herein by reference in its entirety.

### STATEMENT OF FEDERALLY SPONSORED RESEARCH OR DEVELOPMENT

This invention was made with government support under DK126697 and A1158418 awarded by the National Institutes of Health. The government has certain rights in the invention.

### MATERIAL INCORPORATED BY REFERENCE

The Sequence Listing, which is a part of the present disclosure, includes a computer-readable form comprising nucleotide and/or amino acid sequences of the present invention (file name “020923-US-NP_2025-10-04_Sequence-Listing” created on 4 Oct. 2025; 18,185 bytes). The subject matter of the Sequence Listing is incorporated herein by reference in its entirety.

### FIELD

The present disclosure generally relates to mediation of alpha-hemolysin toxicity.

### BACKGROUND

Bacterial toxins are central to pathogenesis and inflict host tissue damage through direct cell injury and by provoking deleterious inflammatory responses. Strains of uropathogenic Escherichia coli (UPEC) encode for multiple secreted toxins that contribute to host inflammation and renal damage, including α-hemolysin (HlyA), a prototypic member of the large bacterial repeats-in-toxin (RTX) family, so named for a conserved GGxGxDxUx sequence repeat. UPEC is the primary cause of urinary tract infections (UTI), both in the bladder (cystitis) or the kidney (pyelonephritis). Pyelonephritis can leave renal scars despite appropriate antibiotic treatment and confers risk for chronic kidney disease later in life.

Carriage of the hlyCABD operon, which encodes for the active toxin (HlyA) as well as modification and delivery components, is more prevalent among UPEC isolates from patients with pyelonephritis than with cystitis. Moreover, there is wide variation in hemolytic activity (i.e., HlyA secretion) among UPEC strains, reflecting complex transcriptional activation and hlyCABD coding polymorphisms. Though recent advances in mouse models have enabled more detailed interrogation of bacterial pathogenesis in the kidney, the influence of HlyA in the pathogenesis of pyelonephritis remains poorly defined.

HlyA is secreted via classical Gram-negative type 1 secretion and can exert cytotoxicity toward a variety of cell types. Typical structural and biochemical characterization of HlyA has been precluded by long-standing challenges in purification and functional analysis, due largely to its amphipathic nature and poor stability. As HlyA can lyse red blood cells (RBCs), and biochemical (though not structural) data indicate it can form pores in erythrocyte membranes, HlyA has long been presumed to function as a pore-forming toxin (PFT) that causes cell death via disruption of osmotic homeostasis. Application of sublytic concentrations of HlyA to epithelial cells led to activation of serine proteases in the cytosol of intoxicated cells (posited to be a downstream effect of plasma membrane poration) and elicited production of cytokines such as IL-6, IL-8, and GM-CSF. HlyA has been studied mostly in myeloid cells, where (as with several other RTX toxins) direct interaction with the leukocyte β2-integrin CD11a/CD18 (LFA-1) is implicated in its cytotoxicity. As healthy epithelial cells lack CD11a/CD18 expression, HlyA must function in ways beyond those currently known. Further, HlyA of E. coli has been recognized for ˜100 years, but its mechanisms of action have been elusive, owing largely to its instability and difficulty with typical protein purification and structure determination.

### BRIEF DESCRIPTION OF THE DISCLOSURE

Among the various aspects of the present disclosure is the provision of hemolysin inhibition/blocking in the context of E. coli urinary tract infections.

In accordance with an aspect of the present disclosure, a composition comprising a soluble low-density lipoprotein receptor (LDLR)-Fc fusion protein is provided. The soluble LDLR-Fc fusion protein comprises: an Fc domain; and at least one LDLR type A domain. In some embodiments, the at least one LDLR type A domain comprises at least one of a ligand binding domain (LBD), LA1, LA2, LA3, LA4, LA5, LA6, LA7, and combinations thereof. In some embodiments, the at least one LDLR type A domain comprises at least one tandem domain selected from LA1-2, LA2-3, LA3-4, LA4-5, LA5-6, LA6-7, and combinations thereof.

In accordance with another aspect of the present disclosure, a method of treating urinary tract infection (UTI) in a subject in need thereof is provided. The method comprises administering to the subject a composition comprising an alpha-hemolysin (HlyA) inhibiting agent. In some embodiments, the HlyA inhibiting agent comprises a soluble low-density lipoprotein receptor (LDLR)-Fc fusion protein comprising: an Fc domain; and at least one LDLR type A domain. In some embodiments, the at least one LDLR type A domain comprises at least one of a ligand binding domain (LBD), LA1, LA2, LA3, LA4, LA5, LA6, LA7, and combinations thereof. In some embodiments, the at least one LDLR type A domain comprises at least one tandem domain selected from LA1-2, LA2-3, LA3-4, LA4-5, LA5-6, LA6-7, and combinations thereof. In some embodiments, the HlyA inhibiting agent comprises a clathrin-mediated endocytosis (CME) inhibitor and/or an anti-LDLR antibody. In certain embodiments, the HlyA inhibiting agent includes a soluble LDLR-Fc fusion protein as described herein, a clathrin-mediated endocytosis (CME) inhibitor, and/or an anti-LDLR antibody. For example, depending upon the embodiment, the HlyA inhibiting agent is includes: a LDLR-Fc fusion protein; a LDLR-Fc fusion protein and a CME inhibitor; a LDLR-Fc fusion protein and an anti-LDLR antibody; or a LDLR-Fc fusion protein, a CME inhibitor, and an anti-LDLR antibody. In some embodiments, administering the HlyA inhibiting agent protects against cell damage. In some embodiments, the UTI is cystitis and the cell damage includes at least one of bladder cell damage and bladder tissue damage. In some embodiments, the UTI is pyelonephritis and the cell damage comprises at least one of renal cell damage and renal tissue damage. In some embodiments, the UTI is caused by E. coli.

In accordance with a further aspect of the present disclosure, a method of treating sepsis in a subject in need thereof is provided. The method comprises administering to the subject a composition comprising an alpha-hemolysin (HlyA) inhibiting agent. In some embodiments, the HlyA inhibiting agent comprises a soluble low-density lipoprotein receptor (LDLR)-Fc fusion protein comprising: an Fc domain; and at least one LDLR type A domain. In some embodiments, the at least one LDLR type A domain comprises at least one of a ligand binding domain (LBD), LA1, LA2, LA3, LA4, LA5, LA6, LA7, and combinations thereof. In some embodiments, the at least one LDLR type A domain comprises at least one tandem domain selected from LA1-2, LA2-3, LA3-4, LA4-5, LA5-6, LA6-7, and combinations thereof. In some embodiments, the HlyA inhibiting agent comprises a clathrin-mediated endocytosis (CME) inhibitor and/or an anti-LDLR antibody. In certain embodiments, the HlyA inhibiting agent includes a soluble LDLR-Fc fusion protein as described herein, a clathrin-mediated endocytosis (CME) inhibitor, and/or an anti-LDLR antibody. For example, depending upon the embodiment, the HlyA inhibiting agent is includes: a LDLR-Fc fusion protein; a LDLR-Fc fusion protein and a CME inhibitor; a LDLR-Fc fusion protein and an anti-LDLR antibody; or a LDLR-Fc fusion protein, a CME inhibitor, and an anti-LDLR antibody. In some embodiments, the sepsis is caused by E. coli.

Other objects and features will be in part apparent and in part pointed out hereinafter.

### BRIEF DESCRIPTION OF THE DRAWINGS

Those of skill in the art will understand that the drawings described herein are for illustrative purposes only. The drawings are not intended to limit the scope of the present teachings in any way.

FIG. 1A is a schematic of experimental procedures and timeline. Triangles indicate testosterone cypionate (or vehicle) dosing; all data in subsequent panels reflect 14 dpi with the indicated UPEC strains.

FIG. 1B is a graph of bacterial loads (CFU) in the kidneys of infected mice (ANOVA not significant [ns]).

FIG. 1C is a set of histology images of mouse kidneys, as visualized by H&E staining (Left column) or IHC for Kim-1 (Right column).

FIG. 1D is a graph of the proportion of the kidney cortex affected by ATN. ANOVA P=0.016; *P=0.025 for hlyA++vs. ΔhlyA:1, P=0.035 for hlyA++vs. ΔhlyA:2 by Tukey post hoc analysis.

FIG. 1E is a graph of the proportion of cortical tubules that stained positive for Kim-1. ANOVA P=0.043; P=0.064 for hlyA++vs. ΔhlyA:2 by Tukey post hoc analysis.

FIG. 1F is a graph of serum Kim-1 measured by ELISA. ANOVA P=0.0082; *P=0.021 for hlyA++vs. ΔhlyA:1, P=0.034 for hlyA++vs. ΔhlyA:2 by Tukey post hoc analysis.

FIG. 2A is a schematic of the experimental approach for the CRISPR-Cas9 screen.

FIG. 2B is a set of Volcano plots displaying log fold change in gene abundance vs. −log 10 P-value (dashed line=FDR threshold of 0.05) after sequential HlyA-CM challenges.

FIG. 2C is a schematic of a string pathway analysis of the top 100 altered genes across the 3 toxin exposures; the 11 genes meeting FDR threshold are indicated in bold text.

FIG. 2D is a graph of the percent cytotoxicity in IMCD-3 cells exposed to HlyA-CM (4 HU) for 2 h after pretreatment with Dyngo-4A or vehicle. ANOVA P<0.0001; *P=0.037, ****P<0.0001 vs. control (0 Dyngo-4a) by the Dunnett multiple comparison test.

FIG. 2E is a graph of the percent cytotoxicity in primary C57BL/6 renal epithelial cells exposed to HlyA-CM for 2 h after pretreatment with Dyngo-4A or vehicle. ***P<0.001, ****P<0.0001 by the unpaired t test.

FIG. 2F is a graph of the relative Cltc expression in IMCD-3 cells 24 h after transfection with Cltc-siRNA or scramble control. **P<0.01 by the unpaired t test.

FIG. 2G is a graph of the percent cytotoxicity in IMCD-3 cells 24 h after transfection with Cltc-siRNA or scramble control, following HlyA-CM exposure for 2 h. *P<0.05, **P<0.01, ***P<0.001, ****P<0.0001 by the unpaired t test. For all cytotoxicity data, individual experiments are shown and are representative of at least three biologically distinct experiments using CM produced on the day of each experiment.

FIG. 3A is a representative flow cytometry trace of LysoTracker Red signal (Left) and quantification of lysosomal integrity (Right) in IMCD-3 cells exposed to increasing concentrations of HlyA-CM or to ΔhlyA-CM. ANOVA P<0.0001; ****P<0.0001 vs. ΔhlyA-CM by the Dunnett multiple comparison test.

FIG. 3B is a graph of increasing pHrodo Green signal in IMCD-3 cells, reflecting decreased cytoplasmic pH, upon treatment with increasing concentrations of HlyA-CM or with ΔhlyA-CM. ANOVA P<0.0001; ****P<0.0001 vs. ΔhlyA-CM by the Dunnett multiple comparison test.

FIG. 3C is a set of immunoblots for lysosomal proteins (LAMP-2, Cathepsin-D) in the cytoplasmic fraction of IMCD-3 cells exposed to HlyA-CM or ΔhlyA-CM. E-cadherin blot demonstrates no membrane contamination of the cytoplasmic fractions.

FIG. 3D is a graph of flow cytometry analyses of IMCD-3 cells 15 min post HlyA-CM or ΔhlyA-CM challenge. Mitochondrial membrane potential as measured by TMRM fluorescence. ANOVA P<0.0001; ****P<0.0001 vs. ΔhlyA-CM by the Dunnett multiple comparison test.

FIG. 3E is a graph of flow cytometry analyses of IMCD-3 cells 15 min post HlyA-CM or ΔhlyA-CM challenge. MitoTracker Red fluorescence. ANOVA P<0.0001; ****P<0.0001 vs. ΔhlyA-CM by the Dunnett multiple comparison test.

FIG. 3F is a graph of flow cytometry analyses of IMCD-3 cells 15 min post HlyA-CM or ΔhlyA-CM challenge. MitoTracker Green fluorescence. ANOVA P<0.0001; *P=0.014, ****P<0.0001 vs. ΔhlyA-CM by the Dunnett multiple comparison test.

FIG. 3G is a graph of IMCD-3 cytotoxicity with HlyA-CM or ΔhlyA-CM exposure, after pretreatment with the indicated concentrations of the pancaspase inhibitor ZVAD-FMK; ANOVA ns. Fluorescence is expressed as geometric mean relative to fluorescence in the ΔhlyA-CM condition. For all cytotoxicity and flow cytometry data, individual experiments are shown and are representative of at least three biologically distinct experiments using CM produced on the day of each experiment.

FIG. 4A is a graph of the percent cytotoxicity in HEK293T cells following transfection with LRPAP1 or LRPAP1+LDLR 24 h prior to HlyA-CM exposure.

FIG. 4B is a graph of qPCR for LDLR expression in HEK293T cells 24 h posttransfection with LRPAP1 or LRPAP1+LDLR.

FIG. 4C is immunoblotting for LDLR expression in HEK293T cells 24 h posttransfection with LRPAP1 or LRPAP1+LDLR.

FIG. 4D is a graph of the percent cytotoxicity in WT or ΔLdir IMCD-3 cells exposed to HlyA-CM for 2 h.

FIG. 4E is a set of immunoblots demonstrating LDLR production in WT vs. ΔLdir IMCD-3 cells.

FIG. 4F is a graph of flow cytometry-based analyses of WT or ΔLdir IMCD-3 cells challenged with HlyA-CM or ΔhlyA-CM as indicated. Lysosomal integrity as measured by LysoTracker Red fluorescence. ANOVA P<0.0001; ***P=0.008 and ****P<0.0001 vs. ΔhlyA-CM by the Dunnett multiple comparison test.

FIG. 4G is a graph of flow cytometry-based analyses of WT or ΔLdir IMCD-3 cells challenged with HlyA-CM or ΔhlyA-CM as indicated; cytoplasmic acidification as measured by pHrodo Green fluorescence.

FIG. 4H is a graph of flow cytometry-based analyses of WT or ΔLdir IMCD-3 cells challenged with HlyA-CM or ΔhlyA-CM as indicated. Mitochondrial membrane potential as measured by TMRM fluorescence. All fluorescence data are expressed as geometric mean relative to that in the ΔhlyA-CM condition. For all cytotoxicity and flow cytometry data, individual experiments are shown and are representative of at least three biologically distinct experiments using CM produced on the day of each experiment. RFU, relative fluorescence units. Comparisons in FIGS. 4A, B, D, G, and H were made using unpaired t tests, with *P<0.05, **P<0.01, ***P<0.001, ****P<0.0001.

FIG. 5A is an immunoblot (Left) for HlyA on cell lysates after treatment of WT or ΔLdir IMCD-3 cells with HlyA-CM; quantification shown at Right (***P<0.001; by the unpaired t test).

FIG. 5B is a graph of the percent cytotoxicity in WT or ΔLdir IMCD-3 cells exposed for 2 h to HlyA-CM (4 HU) with addition of human LDL (at the indicated concentrations) or BSA (control). ANOVA P<0.0001; ****P<0.0001 vs. 0 LDL by the Dunnett multiple comparison test.

FIG. 5C is a graph of the percent cytotoxicity in WT or ΔLdir IMCD-3 cells exposed for 2 h to HlyA-CM (7.5 HU) after addition of APOB-His (at the indicated concentrations) or BSA (control). ANOVA P<0.0001; ****P<0.0001 vs. 0 LDL by the Dunnett multiple comparison test.

FIG. 5D is a graph of the inhibition of HlyA-mediated toxicity to IMCD-3 cells upon treatment with the indicated concentrations of LDLR-Fc fusion protein, compared with an unrelated control antibody (anti-West Nile virus hE16 [αWNV]). At each HlyA-CM concentration, ANOVA P<0.0001, ****P<0.0001 vs. αWNV control by the Dunnett multiple comparison test.

FIG. 5E is a graph of similar inhibition of HlyA-mediated toxicity to primary murine renal epithelial cells upon treatment with LDLR-Fc (1 μg/mL); **P<0.01 by the unpaired t test. For all cytotoxicity data, individual experiments are shown and are representative of at least three biologically distinct experiments using CM produced on the day of each experiment.

FIG. 6A is a schematic representation of design of UT189 strains with differential HlyA secretion capacity, including two independent ΔhlyA clones.

FIG. 6B is a graph of the quantification of hemolytic activity of engineered UT189 HlyA secretion mutants on 5% sheep blood agar (representative images below the graph).

FIG. 6C is a graph of qPCR of hlyA expression by the indicated strains.

FIG. 6D is a graph of qPCR of cnf1 expression (downstream of the hlyCABD operon) by the indicated strains.

FIG. 6E is a graph of growth curves of the strains in LB broth (point symbols omitted for clarity). **p<0.01, ***p<0.001, ****p<0.0001 by ANOVA with Dunnett multiple comparison test.

FIG. 7A is a graph of bacterial loads in the bladder at the indicated days post infection (dpi) following UPEC infection of androgen-exposed female C57BL/6 mice. Day 14 kidney bacterial loads are the same as shown in FIG. 1B. CFU, colony-forming units. n=5-18 mice per condition at each time point; no significant differences between infecting strains by ANOVA.

FIG. 7B is a graph of bacterial loads in the kidney at the indicated days post infection (dpi) following UPEC infection of androgen-exposed female C57BL/6 mice. Day 14 kidney bacterial loads are the same as shown in FIG. 1B. CFU, colony-forming units. n=5-18 mice per condition at each time point; no significant differences between infecting strains by ANOVA.

FIG. 8A is a schematic overview of HlyA-CM preparation.

FIG. 8B is a graph of percent cytotoxicity in IMCD3, BMDM, J774, and HEK293T cells exposed to HlyA-CM (1-22 HU) for 2 h.

FIG. 8C is graph of minimal cytotoxicity of ΔhlyA-CM to various cell lines (note y-axis range).

FIG. 8D is graph showing verification of CRISPR mutant library coverage (79,633 gRNAs).

FIG. 9 is a graph comparing p-values to fold-change for GO PANTHER pathway terms representing the guides enriched in surviving CRISPR-modified IMCD-3 cells.

FIG. 10A is a graph of fluorescent transferrin (Tf) uptake in IMCD-3 cells treated with the dynamin inhibitor Dyngo-4A or with DMSO control. Single experiment with technical replicates shown. RLU, relative luminescence units. ***p<0.001 by un-paired t-test.

FIG. 10B is a graph of membrane cholesterol in IMCD-3 cells treated with the dynamin inhibitor Dyngo-4A or with DMSO control. Single experiment with technical replicates shown. RLU, relative luminescence units. ***p<0.001 by un-paired t-test.

FIG. 11A is a graph of membrane cholesterol levels in IMCD-3 cells receiving siRNA targeting Cltc or Ap2m1, or scramble siRNA control (single experiment with technical replicates; ns by ANOVA vs Scramble). RLU, relative luminescence units.

FIG. 11B is a graph of knockdown of Ap2m1 mRNA, measured by qPCR; p value as shown by unpaired t test.

FIG. 11C is a graph showing reduced HlyA cytotoxicity to IMCD-3 cells with siRNA knockdown of Ap2m1. p values as shown and **p<0.01 by unpaired t-test.

FIG. 12A is a set of representative immunofluorescence microscopy images of cells exposed to ΔhlyA-CM or 2.5-10 HU of HlyA-CM, stained with wheat germ agglutinin (WGA; red) and DAPI (blue).

FIG. 12B is a set of representative histograms of IMCD-3 cells treated with HlyA-CM and analyzed by flow cytometry for pHrodo Green (see FIG. 3B), TMRM (FIG. 3D), MitoTracker Red (FIG. 3E), and MitoTracker Green (FIG. 3F).

FIG. 12C is a graph showing pan-caspase inhibitor Z-VAD-FMK inhibited staurosporine-induced apoptosis of IMCD-3 cells, compared with 0.01% DMSO (vehicle) (control for FIG. 3G). **p<0.01, ***p<0.001 by ANOVA with Dunnett multiple comparison test.

FIG. 13A is a graph of membrane cholesterol levels in HEK293T cells after transfection with LRPAP1 or LRPAP1+LDLR.

FIG. 13B is a graph of membrane cholesterol levels in wild-type (WT) IMCD-3 cells and cells with CRISPR-mediated disruption of Ldlr. Single experiment with technical replicates shown. RLU, relative luminescence units.

FIG. 14A is a graph of CRISPR disruption of Ldlr in a second, independent clone of IMCD-3 cells confirms protection from HlyA-mediated cytotoxicity. ****p<0.0001 by unpaired t test.

FIG. 14B is a set of representative histograms of WT (upper traces) and ΔLdir (lower traces) IMCD-3 cells treated with HlyA-CM and analyzed by flow cytometry for LysoTracker Red (see FIG. 4F), pHrodo Green (FIG. 4G), and TMRM (FIG. 4H).

FIG. 15 is a table of statistically significant genes in the CRISPR mutant screen.

FIG. 16 is a table of oligonucleotides used in the Example 1 study.

FIG. 17 is a schematic of the structure of an a/P integrin in high-affinity conformation. The β2 integrin CD18 heterodimerizes with one of four a subunits; common names for these dimers are shown.

FIG. 18 is a schematic of the domain structure of LDLR (mouse or human). LBD, ligand-binding domain.

FIG. 19 is a pair of images of colonies of hemolytic wild-type (WT) CFT073 and the isogenic ΔhlyA mutant on 5% sheep blood agar.

FIG. 20A is a survival curve of male C57BL/6 mice after retroorbital inoculation with 108 CFU CFT073 (open circles) or CFT073 ΔhlyA (filled circles); p=0.016 by Mantel-Cox log-rank test. hpi, hours post infection.

FIG. 20B is a graph of weight loss, shown as percentage decrease from starting weight, in male C57BL/6 mice surviving 48 h after retroorbital inoculation with 108 CFU UPEC CFT073 (open circles) or CFT073 ΔhlyA (filled circles); p=0.002 by unpaired t test.

FIG. 21 is a graph of dose-dependent killing of myeloid cells by HlyA. Cell death (as measured by LDH release) of U937 cells (blue squares) or THP-1 cells (green circles) after 45 min exposure to HlyA-CM (left panel), or to ΔhlyA-CM (bars, right panel). HU, hemolytic units.

FIG. 22 is a graph showing HlyA toxicity to myeloid cell lines requires endocytosis. Pre-treatment with the dynamin inhibitor Dyngo-4a abrogated toxicity of HlyA-CM to U937 (blue) and THP-1 cells (green). p values shown (unpaired t tests).

FIG. 23 is a graph showing dose-dependent killing of EAhy.926 cells by HlyA. Cell death (measured by LDH release) upon exposure to indicated concentrations of HlyA or to ΔhlyA-CM. HU, hemolytic units. ****p<0.0001 by ANOVA with Dunnett multiple comparison test, vs. ΔhlyA-CM.

FIG. 24A is a graph of blood and organ bacterial loads 24 h into CFT073 sepsis. Organ titers shown as CFU per mL of blood. Dashed lines denote lower limit of detection.

FIG. 24B is a graph of blood and organ bacterial loads 24 h into CFT073 sepsis. Organ titers shown as CFU per g of liver. Dashed lines denote lower limit of detection.

FIG. 24C is a graph of blood and organ bacterial loads 24 h into CFT073 sepsis. Organ titers shown as CFU per kidney. Dashed lines denote lower limit of detection.

FIG. 24D is a graph of blood and organ bacterial loads 24 h into CFT073 sepsis. Organ titers shown as CFU per lung. Dashed lines denote lower limit of detection.

FIG. 25 is a table of a list of mice for conditional deletion of Ldlr and/or Itgb2 in ECs or myeloid cells.

### DETAILED DESCRIPTION OF THE DISCLOSURE

The present disclosure is based, at least in part, on the discovery that mediation of alpha-hemolysin (HlyA) toxicity via cellular pathways and testosterone is significant for treating urinary tract infections and for preventing/minimizing cell and tissue damage during infection.

The present disclosure demonstrates mouse models of E. coli pyelonephritis that HlyA causes damage to renal tissue, with no direct effect on colonization (i.e., the raw bacterial loads in the kidney). Moreover, herein is identified for the first time that the cytotoxicity of HlyA to renal epithelial cells requires clathrin-mediated endocytosis and the presence of the low-density lipoprotein receptor (LDLR). These findings arose from a CRISPR mutant screen in cultured renal epithelial cells; confirmatory data include (i) pharmacologic agents that impair CME also protect renal epithelial cells from HlyA toxicity; (ii) provision of a soluble LDLR-Fc fusion protein protects renal epithelial cells from HlyA toxicity, presumably by acting as a decoy receptor (or “sink”) for HlyA.

LDLR is also used as an entry receptor for a number of viruses, particularly in the alphavirus family; the inventors have previously created LDLR-Fc and related constructs, some of which block entry of certain viruses into cells, and can protect mice from certain fatal viral infections. This concept (potential use of LDLR-Fc and derivatives as therapeutics in selected alphavirus infections) has previously been disclosed by the inventors. Herein a completely different use of the LDLR-Fc and related constructs is demonstrated, namely, to block action of HlyA during E. coli infections of the kidney.

Advantages of the present disclosure include potential adjunctive therapy for E. coli pyelonephritis (in addition to traditional antibiotics), to prevent renal tissue damage associated with infection, and thereby preventing renal scarring and other long-term complications of renal scarring (risk for hypertension, CKD).

### Modulation Agents

As described herein, gene and/or associated protein expression has been implicated in various diseases, disorders, and conditions. As such, modulation of gene and protein expression can be used for treatment of such conditions. A modulation agent can modulate response, such as by inducing or inhibiting gene and/or protein expression signaling. Modulation can comprise modulating protein expression on cells, modulating the quantity of gene/protein expressing cells, or modulating the quality of gene/protein expressing cells.

Modulation agents can be any composition or method that modulates expression on cells. For example, a modulation agent can be an activator, an inhibitor, an agonist, or an antagonist. As another example, the modulation can be the result of gene editing.

A modulation agent can be an antibody (e.g., a monoclonal antibody). A modulating agent can be an agent that induces or inhibits progenitor cell differentiation into gene/protein expressing cells.

Signal Reduction, Elimination, or Inhibition by Small Molecule Inhibitors, shRNA, siRNA, or ASOs As described herein, a modulation and/or inhibiting agent can be used for use in various therapies. A modulation agent can be used to reduce/eliminate or enhance/increase cellular pathway signals. For example, a modulation agent can be a small molecule inhibitor. As another example, a modulation agent can be a short hairpin RNA (shRNA). As another example, a modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g., Zhou et al. 2016 Methods Mol Biol. 1402:199-213. Except as otherwise noted herein, therefore, the process of the present disclosure can be carried out in accordance with such processes.

### Inhibiting Agent

One aspect of the present disclosure provides for targeting of alpha-hemolysin (HlyA), its receptor, or its downstream signaling. The present disclosure provides methods of treating or preventing UTIs and UTI-related cell/tissue damaged based on the discovery that HlyA toxicity can be mediated via cellular pathways (and additionally testosterone in some embodiments).

As described herein, inhibitors or antagonists (e.g., antibodies, fusion proteins, small molecules) can reduce or prevent signaling, binding, activation and/or function. An inhibiting agent can be any agent that can inhibit activity, inhibit signaling, downregulate protein level, downregulate expression, or knockdown gene expression.

For example, the inhibiting agent can be an anti-LDLR antibody or an anti-HlyA antibody, wherein the anti-type antibody prevents binding to a receptor, or prevents activation of downstream signaling. Furthermore, the antibody can be a murine antibody, a humanized murine antibody, or a human antibody.

As another example, the inhibiting agent can be a fusion protein, such as an LDLR-Fc fusion protein. For example, the fusion protein can be a decoy receptor for LDLR. Furthermore, the fusion protein can comprise a mouse or human Fc antibody domain fused to an ectodomain.

As another example, an inhibiting agent can be a clathrin-mediated endocytosis (CME) inhibitor, which has been shown to be a potent and specific inhibitor of HlyA toxicity signaling.

As another example, an inhibiting agent can be an inhibitory protein such as an antagonist. For example, the inhibiting agent can be a viral protein antagonist. As another example, an inhibiting agent can be a short hairpin RNA (shRNA) or a short interfering RNA (siRNA). As another example, an inhibiting agent can be a single guide RNA (sgRNA).

Methods for preparing an inhibiting agent, such as the HlyA inhibiting agent described herein (e.g., an agent capable of inhibiting pathway signaling) can comprise construction of a protein/Ab scaffold containing the natural receptor as a neutralizing agent; developing inhibitors of a receptor “down-stream”; or developing inhibitors of production “up-stream”.

Inhibiting can be performed by genetically modification in a subject or genetically modifying a subject to reduce or prevent expression of a target gene, such as through the use of CRISPR-Cas9 or analogous technologies.

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Chemical Agent

Examples of alpha-hemolysin (HlyA) inhibiting agents are described herein. Agents can include one or more soluble low-density lipoprotein receptor (LDLR)-Fc fusion proteins, clathrin-mediated endocytosis (CME) inhibitors, or anti-LDLR antibodies, and pharmaceutically acceptable salts thereof. Solvates, polymorph, tautomer, prodrug, analog, or stereoisomer thereof or optionally substituted analog thereof are also included.

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀ cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O— isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O— cyclopropyl, —O-cyclobutyl, —O-cyclopentyl, —O-cyclohexyl, —O-cycloheptyl, —O-cyclooctyl, —O— cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated.

The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | |
| (Acidic): | D E |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, |
| | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, |
| | Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, gene and/or protein expression signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of gene/protein expression/signaling by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies are rapidly advancing.

There has recently been an improved landscape for gene therapies. For example, in the first quarter of 2019, there were 372 ongoing gene therapy clinical trials (Alliance for Regenerative Medicine, May 9, 2019).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

Gene therapy strategies.

| | Strategy |
|---|---|
| Viral Vectors | |
| Retroviruses | Retroviruses are RNA viruses transcribing |
| | their single-stranded genome into a double- |
| | stranded DNA copy, which can integrate into |
| | host chromosome |
| Adenoviruses (Ad) | Ad can transfect a variety of quiescent and |
| | proliferating cell types from various species |
| | and can mediate robust gene expression |
| Adeno-associated | Recombinant AAV vectors contain no viral |
| Viruses (AAV) | DNA and can carry ~4.7 kb of foreign |
| | transgenic material. They are replication |
| | defective and can replicate only while |
| | coinfecting with a helper virus |
| Non-viral vectors | |
| plasmid DNA | pDNA has many desired characteristics as a |
| (pDNA) | gene therapy vector; there are no limits on |
| | the size or genetic constitution of DNA, it |
| | is relatively inexpensive to supply, and |
| | unlike viruses, antibodies are not generated |
| | against DNA in normal individuals |
| RNAi | RNAi is a powerful tool for gene specific |
| | silencing that could be useful as an enzyme |
| | reduction therapy or means to promote read- |
| | through of a premature stop codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided are processes of treating, preventing, or reversing urinary tract infection (UTI) and the effects thereof in a subject in need thereof via administration of a therapeutically effective amount of HlyA inhibiting agent, so as to substantially inhibit, slow the progress of, or limit the development of urinary tract infections and cell/tissue damaged caused therefrom.

Methods described herein are generally performed on a subject in need thereof. A subject in need of the therapeutic methods described herein can be a subject having, diagnosed with, suspected of having, or at risk for developing UTIs, including UTIs caused by uropathogenic Escherichia coli (UPEC). A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a HlyA inhibiting agent is, for example, an amount that would cause the desired therapeutic effect in a subject while minimizing undesired side effects. In various embodiments, an effective amount of HlyA inhibiting agent(s) described herein can substantially inhibit, slow the progress of, or limit the development of urinary tract infections and cell/tissue damage caused therefrom.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of HlyA inhibiting agent(s) can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat urinary tract infection and protect against (e.g., prevent or minimize) cell/tissue damage during urinary tract infection.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes reversing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of HlyA inhibiting agent(s) can occur as a single event or over a time course of treatment. For example, HlyA inhibiting agent(s) can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for urinary tract infections, including cystitis (infection of the urinary bladder) and pyelonephritis (infection of the kidney).

A HlyA inhibiting agent can be administered simultaneously or sequentially with another agent, such as an antibiotic, an anti-inflammatory, or another agent. For example, a HlyA inhibiting agent can be administered simultaneously with another agent, such as an antibiotic or an anti-inflammatory. Simultaneous administration can occur through administration of separate compositions, each containing one or more of a HlyA inhibiting agent, an antibiotic, an anti-inflammatory, or another agent. Simultaneous administration can occur through administration of one composition containing two or more of a HlyA inhibiting agent, an antibiotic, an anti-inflammatory, or another agent. A HlyA inhibiting agent can be administered sequentially with an antibiotic, an anti-inflammatory, or another agent. For example, a HlyA inhibiting agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$H E D ( mg / kg ) = Animal dose ( mg / kg ) \times ( Animal {K}_{m} / {Human}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the HlyA inhibiting agent may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a HlyA inhibiting agent such as those described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character x log P of about −2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to HlyA inhibiting agents, precursors, compositions, or formulations thereof, testosterone inhibitors, soluble LDLR-Fc fusion proteins, etc., as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—LDL Receptor-Mediated Endocytosis of Escherichia coli α-Hemolysin Mediates Renal Epithelial Toxicity

The α-hemolysin (HlyA) of uropathogenic Escherichia coli (UPEC) is a pore-forming toxin (PFT) that is thought to function by disrupting the host cell plasma membrane. Although CD18 (LFA-1) has been implicated as a receptor on myeloid cells, the mechanisms underlying HlyA cytotoxicity to epithelial cells are poorly defined. In Example 1, it is shown that HlyA secretion by UPEC markedly intensifies renal tubular epithelial injury in a murine model of ascending pyelonephritis. A CRISPR-Cas9 loss-of-function screen in renal collecting duct cells revealed an unexpected requirement for clathrin-mediated endocytosis in HlyA-induced cytotoxicity. Following internalization, HlyA triggered lysosomal permeabilization, resulting in protease leakage, cytoplasmic acidification, and mitochondrial impairment, culminating in rapid epithelial cell death—a pathway distinct from canonical membrane-disrupting mechanisms of other PFTs. Moreover, the low-density lipoprotein receptor (LDLR) is identified as a critical epithelial receptor for HlyA; genetic ablation or competitive inhibition of the HlyA-LDLR interaction fully abrogated cytotoxicity. The findings in Example 1 detail a paradigm for HlyA function in which epithelial toxicity relies on LDLR-mediated endocytic uptake rather than plasma membrane poration. These mechanistic insights illuminate potential therapeutic strategies to attenuate HlyA-mediated tissue damage during UPEC infections.

More specifically, leveraging an updated preclinical mouse model of ascending pyelonephritis, it is shown that HlyA acts to exacerbate renal tubular injury in vivo. Utilizing a CRISPR-Cas9 loss-of-function screen, it is shown that HlyA toxicity to epithelial cells requires clathrin-mediated endocytosis, representing a mechanism distinct from the proposed model of plasma membrane poration. Instead, HlyA triggers lysosomal permeabilization, leading to rapid cytoplasmic acidification and protease release, with ensuing mitochondrial dysfunction and caspase-independent cell death. Furthermore, the low-density lipoprotein receptor (LDLR) is identified as an HlyA receptor. Genetic disruption of Ldlr reduced HlyA binding to murine renal epithelial cells in vitro and blocked HlyA-induced cell death. Introduction of a soluble LDLR ectodomain fusion peptide (LDLR-Fc) or competition with exogenous LDL or ApoB peptide effectively neutralized HlyA toxicity against renal epithelial cells. The experiments in Example 1 identify a distinct mechanism by which HlyA interacts with and intoxicates epithelial cells, illuminating a novel target for mitigating UPEC-associated renal injury during UTI.

### Results

### α-Hemolysin Secretion Enhances Renal Epithelial Injury During Ascending UTI.

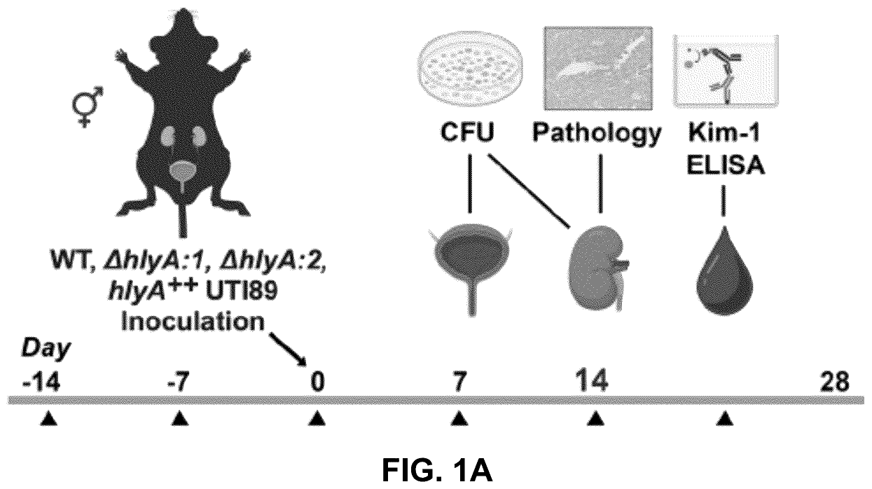

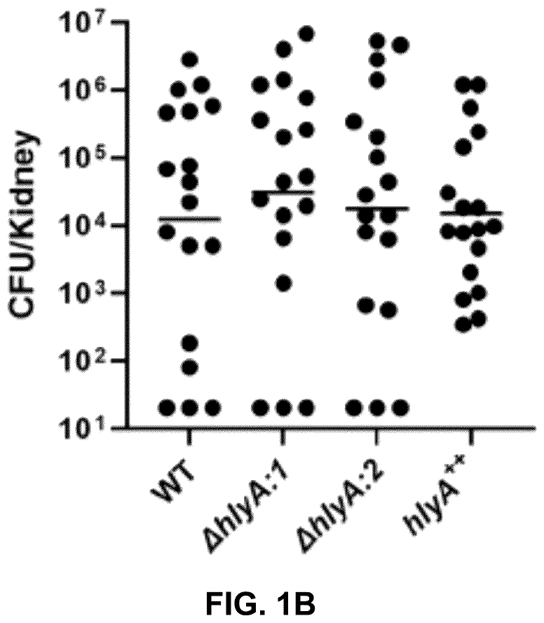

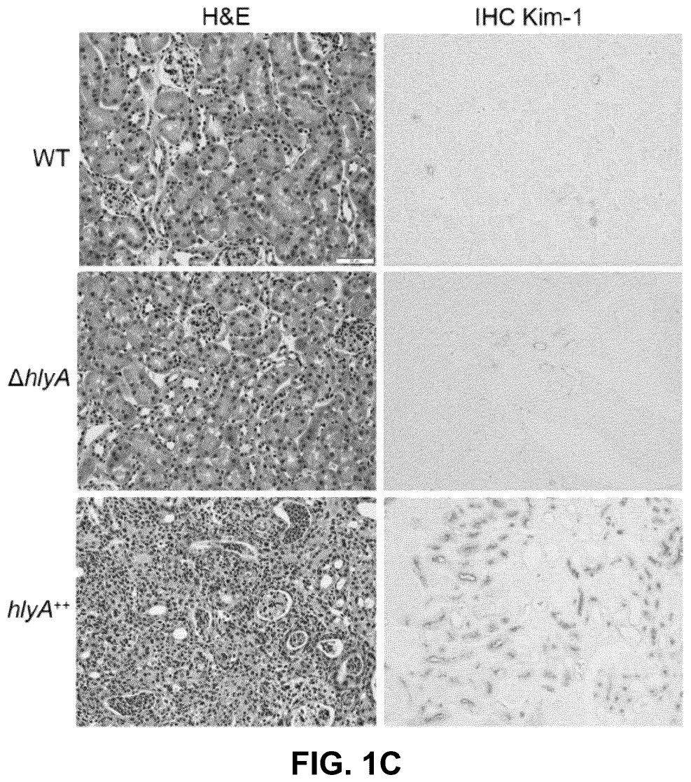

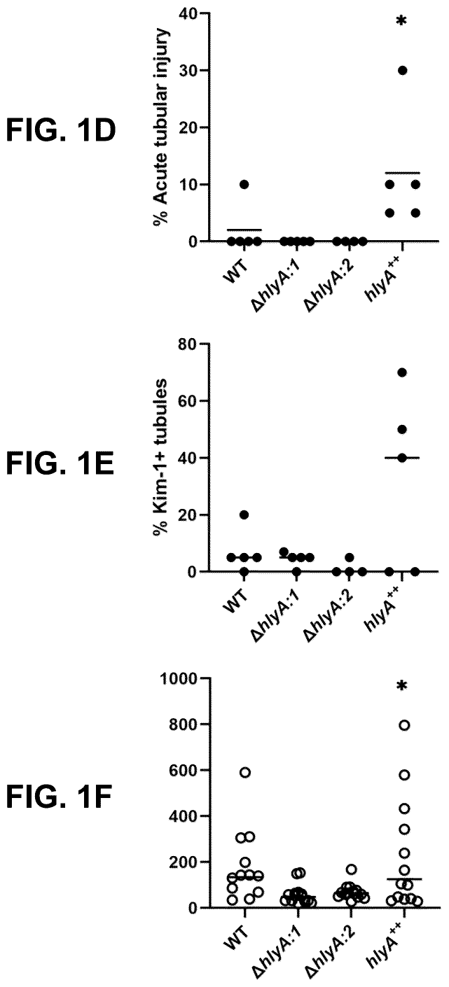

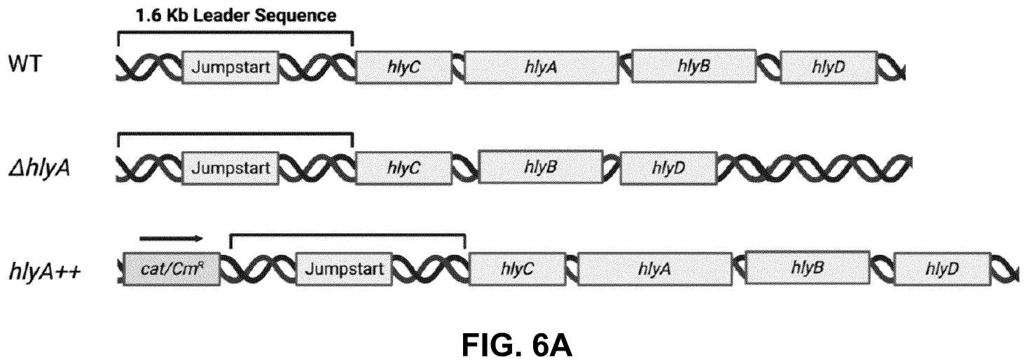

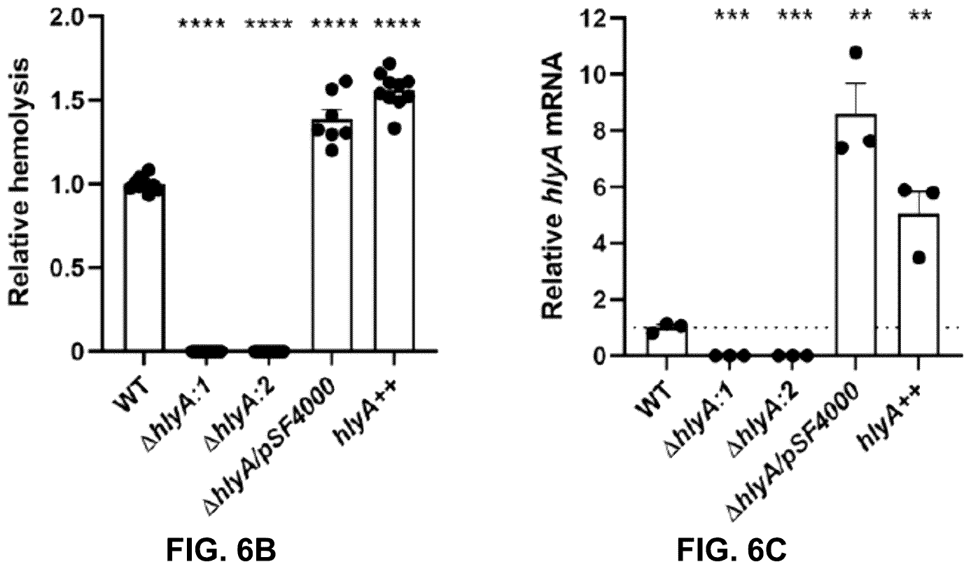

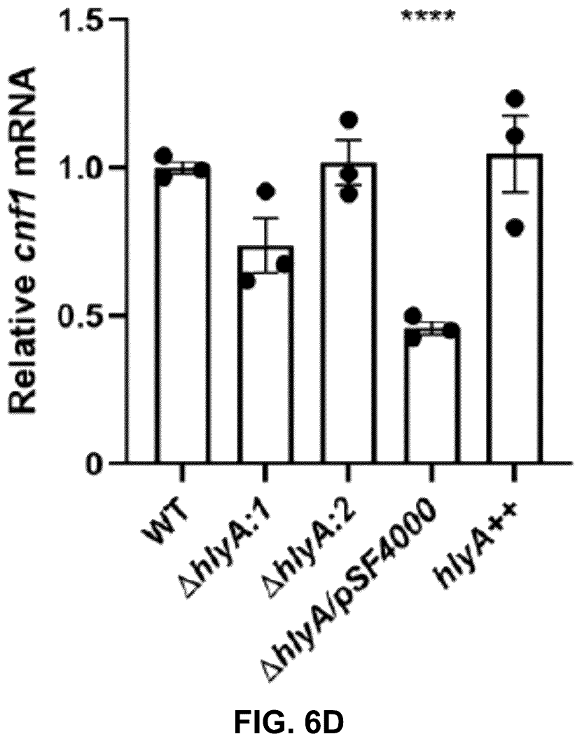

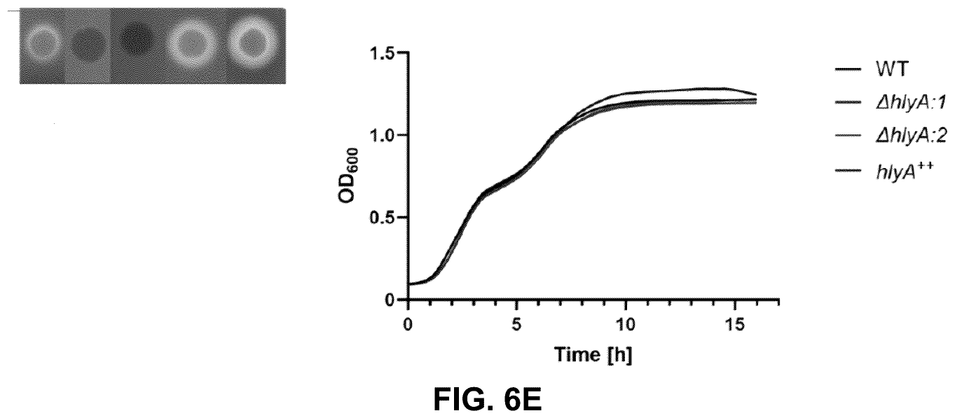

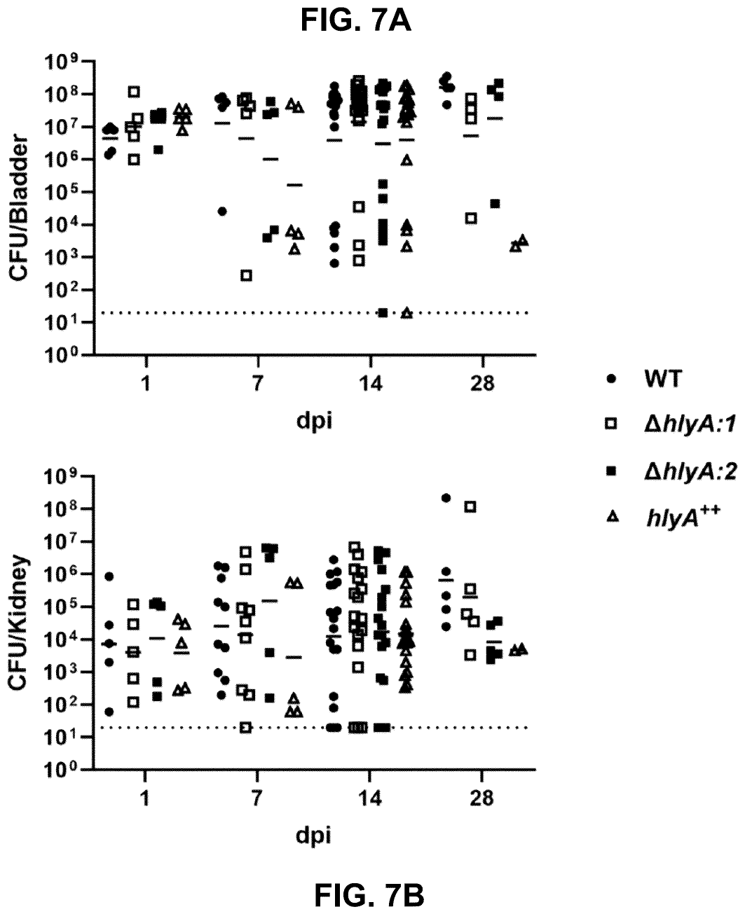

During ascending pyelonephritis, UPEC travel from the bladder through the ureter to access the renal collecting system and colonize the renal tubular space, provoking a local inflammatory response that contributes to renal tissue injury; this disease pathogenesis sequence has been modeled in mice. To determine the contribution of α-hemolysin to renal injury during pyelonephritis, independent hlyA disruption mutants (ΔhlyA:1, ΔhlyA:2) and a chromosomally based HlyA-overexpressing strain (hlyA++) were generated in the UPEC isolate UT189 (FIG. 6A). Genetic alterations in HlyA production were confirmed via quantification of RBC hemolysis and hlyA mRNA expression (FIGS. 6B and C). Furthermore, expression of cnf1, encoding a secreted A-B toxin and positioned downstream of the hlyCABD operon, was not significantly altered in these strains (FIG. 6D), and growth in rich medium was normal (FIG. 6E). These mutant strains or wild-type (WT) UT189 were transurethrally inoculated into the bladders of female C57BL/6 mice [with prior exposure to testosterone, which promotes kidney infection in this background], which were later killed at several time points for histological analyses and bacterial load determination (FIG. 1A). Consistent with prior studies, genetic deletion of hlyA had no significant impact on UPEC colonization of the kidneys (FIG. 1B) or bladder (FIG. 7). Microscopic examination of kidney sections 14 days post infection (dpi) revealed more acute tubular necrosis (ATN) in mice infected with hlyA++UT189 than those infected with WT or ΔhlyA UPEC (FIGS. 1C and D). Furthermore, analysis of kidney sections obtained 14 dpi revealed increased expression of the well-defined renal epithelial injury biomarker Kim-1 in tubules of hlyA++-infected mice (FIGS. 1C and E). Also, compared with WT UT189- or hlyA++-infected mice, mice inoculated with ΔhlyA:1 or ΔhlyA:2 exhibited a significant decrease in circulating Kim-1 protein at 14 dpi (FIG. 1F). These results demonstrate that independent of bacterial burden, HlyA secretion by UPEC enhances renal epithelial damage during pyelonephritis.

### A Genome-Wide CRISPR-Cas9 Screen Identifies Endocytosis as Essential for HlyA Toxicity to Renal Epithelial Cells.

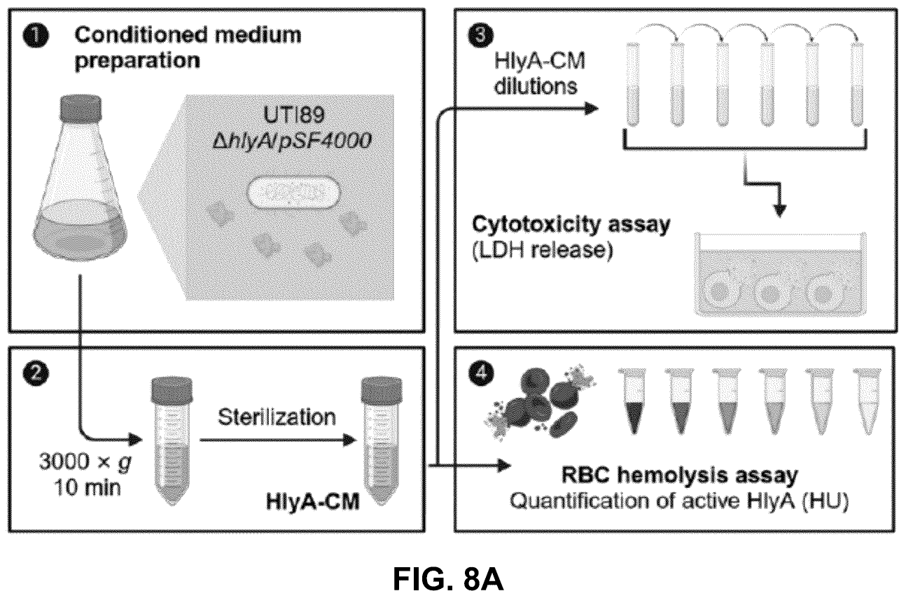

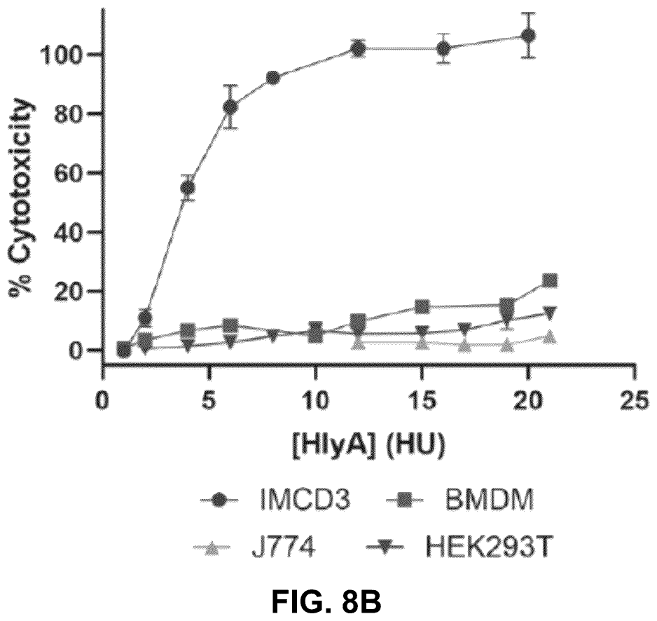

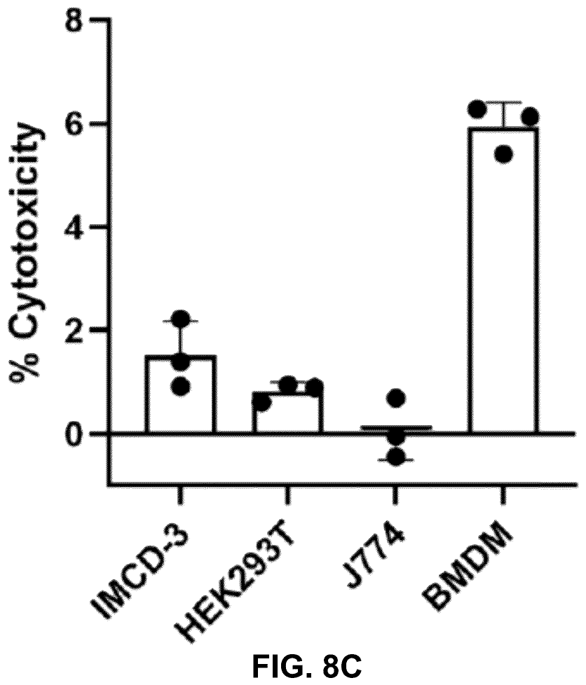

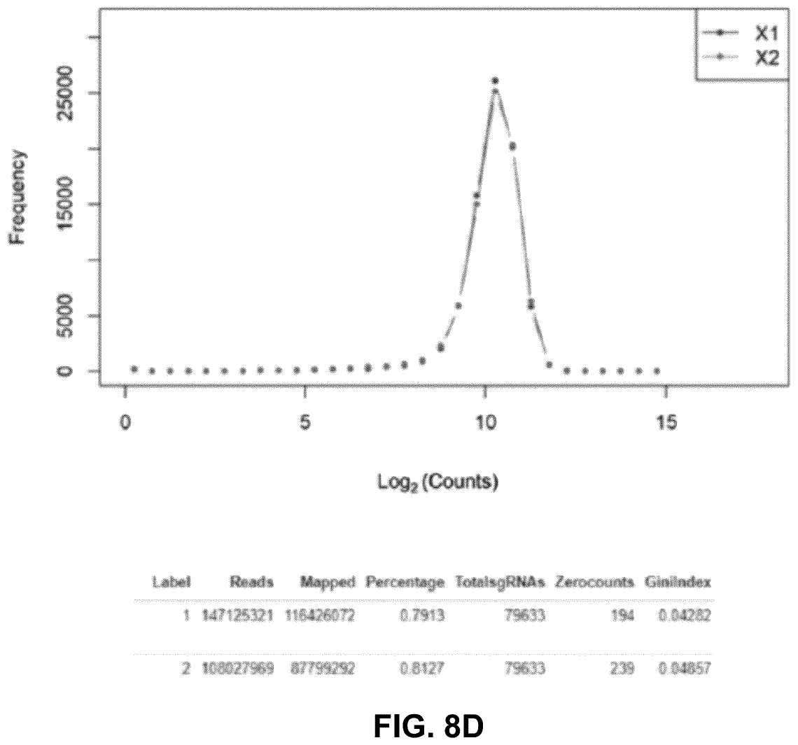

Although in vitro studies have established that HlyA is cytotoxic to renal epithelial cells, an epithelial receptor for HlyA has not been identified. UT189 ΔhlyA transformed with the hlyCABD expression plasmid pSF4000 were grown in LB broth daily and this medium was filter-sterilized prior to its prompt use in experiments (FIG. 8A). The HlyA content of this conditioned medium (HlyA-CM) across daily preparations was standardized by quantitative hemolysis of rabbit RBCs and expressed in arbitrary hemolytic units (HU). Importantly, given this modest daily variation in obtained HlyA concentrations, some figures reflect representative cell-culture experiments (each with several replicate wells) from among several experiments performed on different days. Among multiple cell lines tested, murine inner medullary collecting duct cells (IMCD-3) exhibited substantial cytotoxicity upon HlyA-CM treatment, in contrast to HEK293T (human embryonic kidney fibroblast-like) cells, J774 (murine macrophage-like) cells, or C57BL/6 mouse bone marrow-derived macrophages (BMDMs), which displayed limited susceptibility to HlyA-CM over the tested concentration range (FIG. 8B); of note, J774 and BMDMs express CD18, the reported myeloid-cell receptor for HlyA. Cytotoxicity to any of these lines was low upon treatment with CM prepared identically from UT189 ΔhlyA (FIG. 8C).

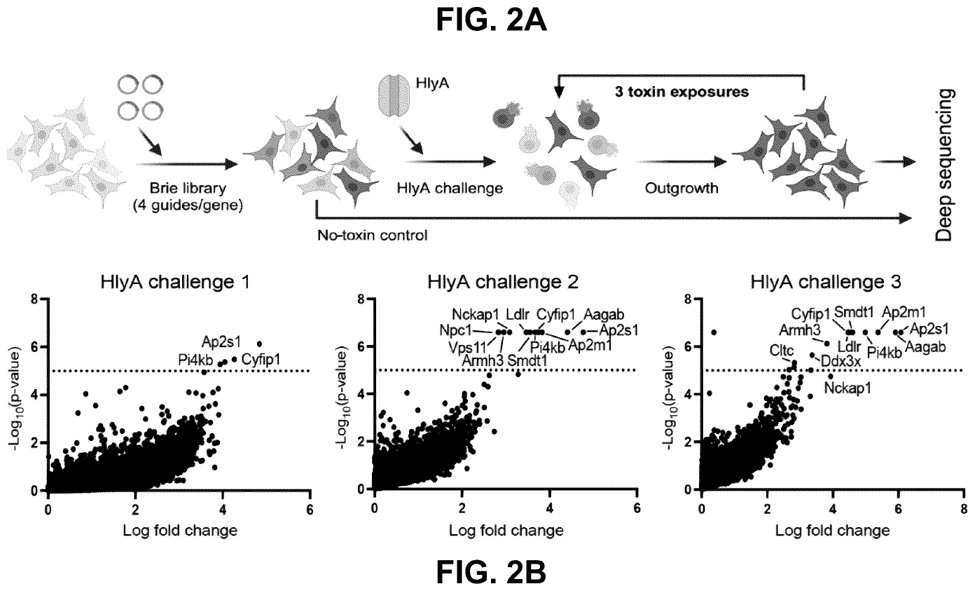

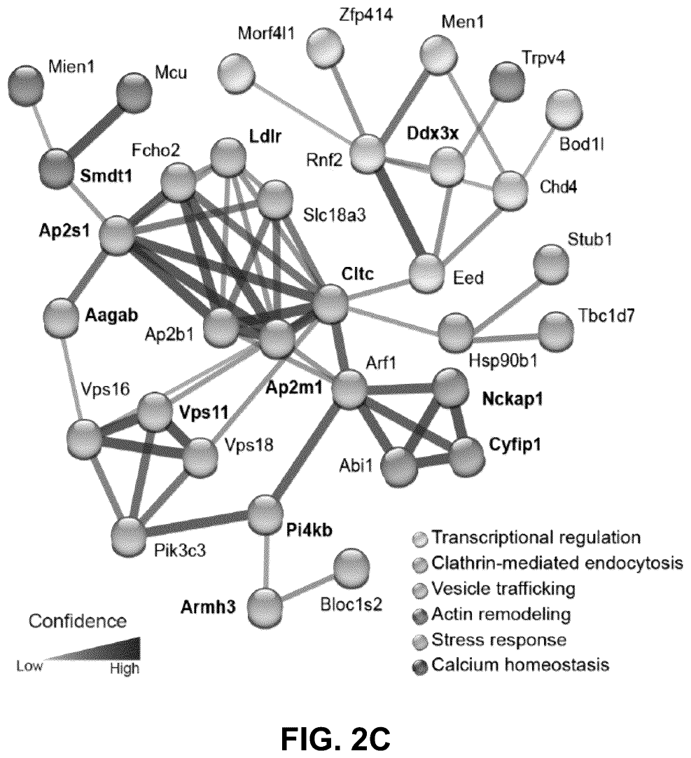

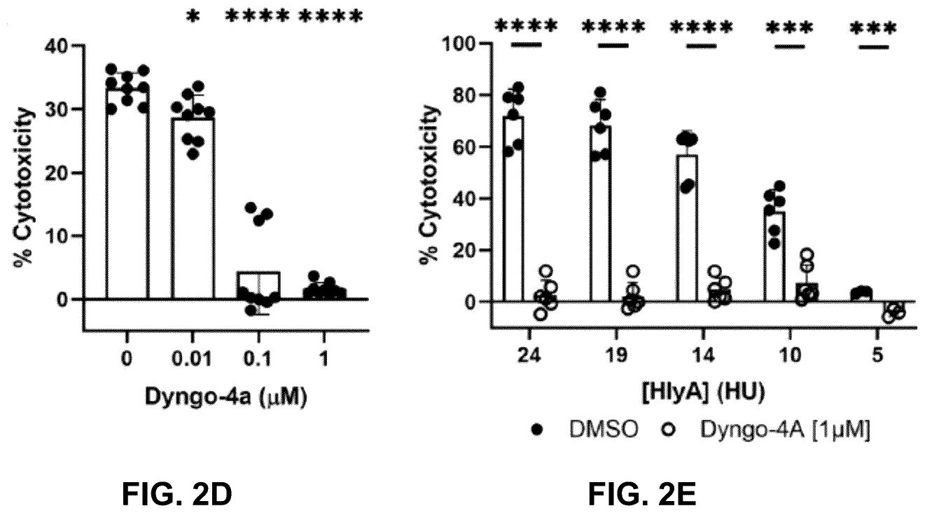

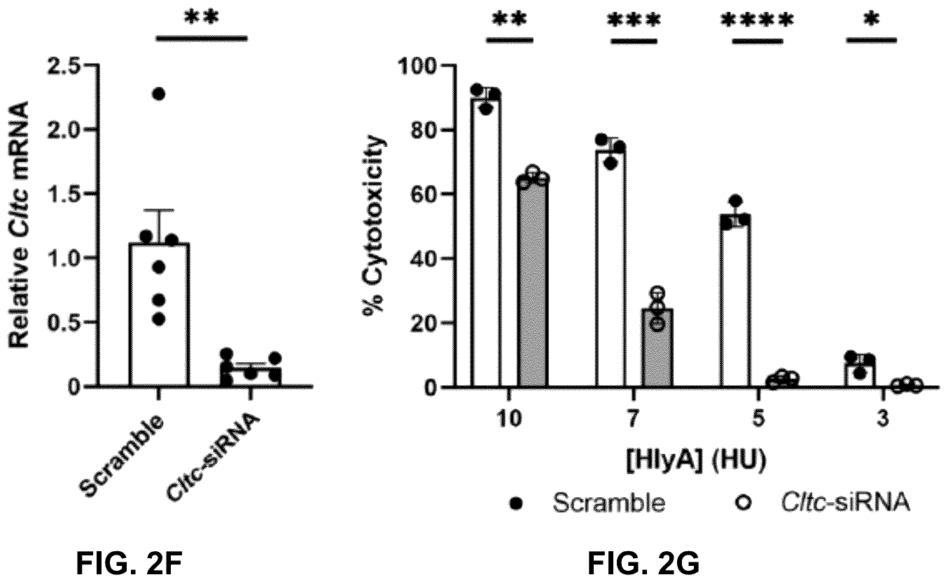

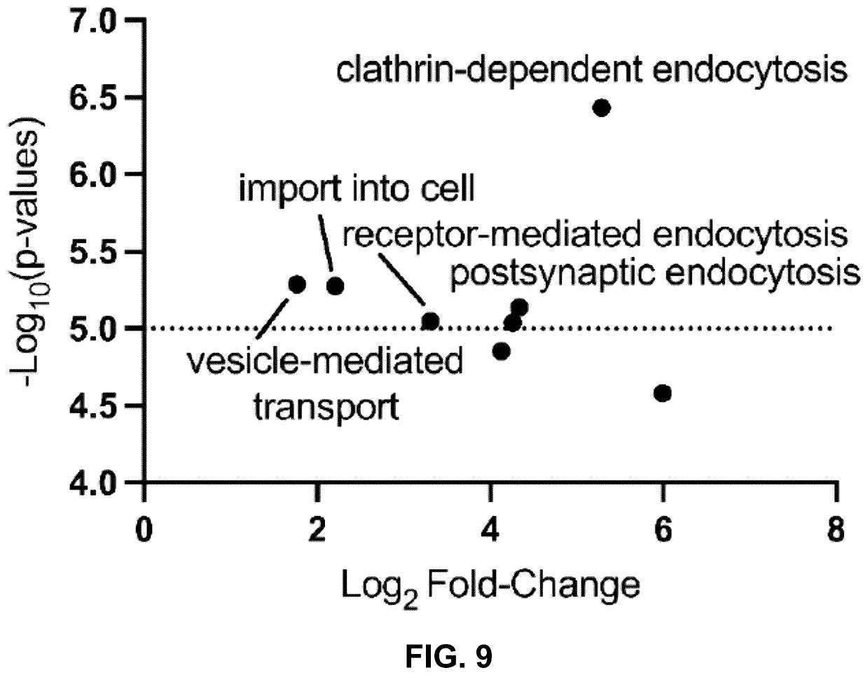

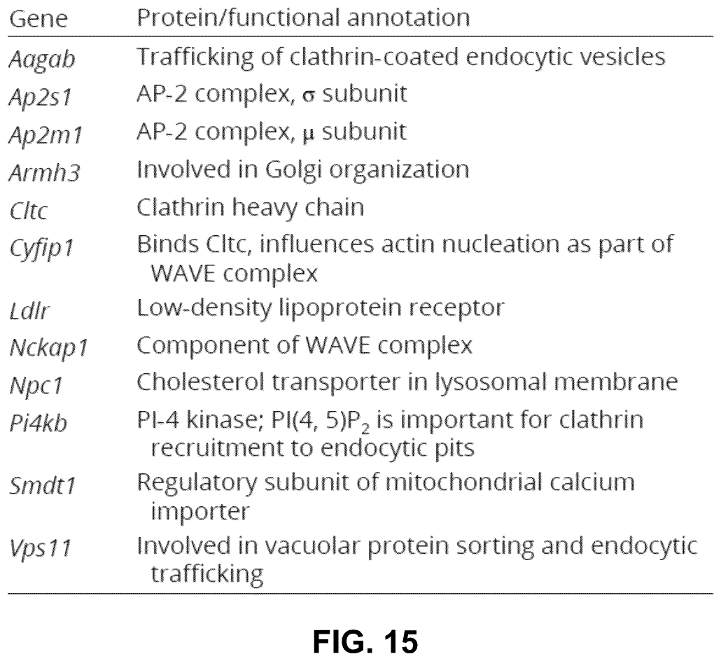

Previously, a CRISPR-Cas9-based gene disruption library was generated in IMCD-3 cells, representing ˜4× nominal coverage of the entire mouse genome (FIG. 8D). To identify host factors required for HlyA toxicity, a survival-based, positive selection screen was performed (FIG. 2A). The library of CRISPR-modified IMCD-3 cells was subjected to HlyA-CM (10 HU) for 2 h to produce ˜60% cell death; surviving cells were then expanded for 48 h. This exposure and recovery cycle was repeated 3 times, with surviving cells collected after each exposure and analyzed by deep sequencing to identify overrepresented sgRNA in surviving cells. The most significant gene “hits” over 3 toxin exposures were Ap2s1, Aagab, and Ap2m1 (FIG. 2D), all of which encode components of the AP-2 adapter complex that links clathrin to the nascent membrane pit to initiate clathrin-mediated endocytosis. Other genes that reached statistical significance in the screen included additional ones participating in clathrin-mediated endocytosis, as well as the low-density lipoprotein receptor (Ldlr) (FIGS. 2B and 15). String and GO PANTHER term analysis of the top 100 aggregate enriched genes over toxin exposures 2 and 3 showed strong relatedness in function and conserved roles in clathrin-mediated endocytosis and vesicle transport (FIGS. 2C and 9).

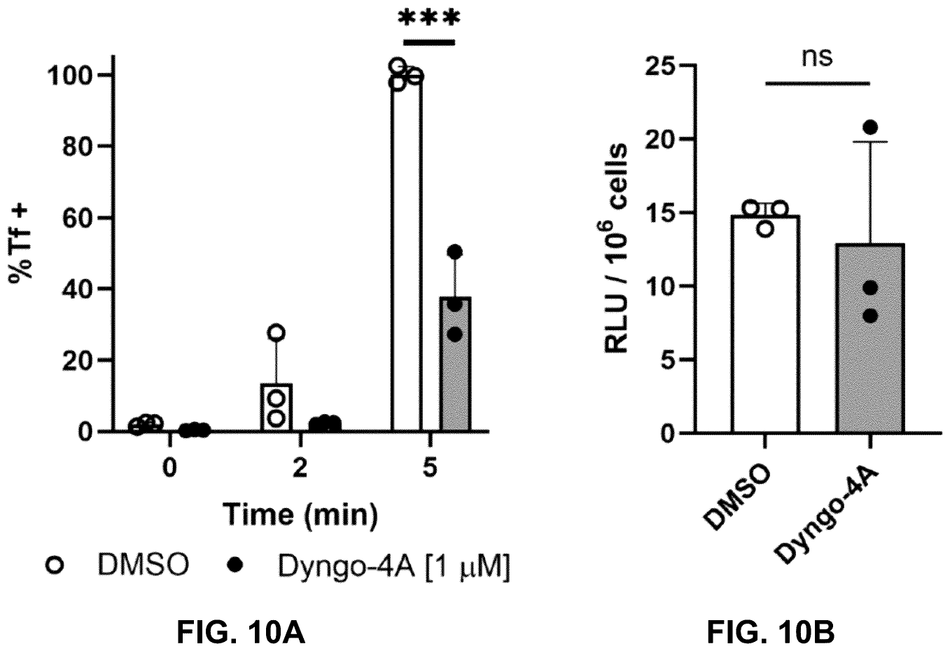

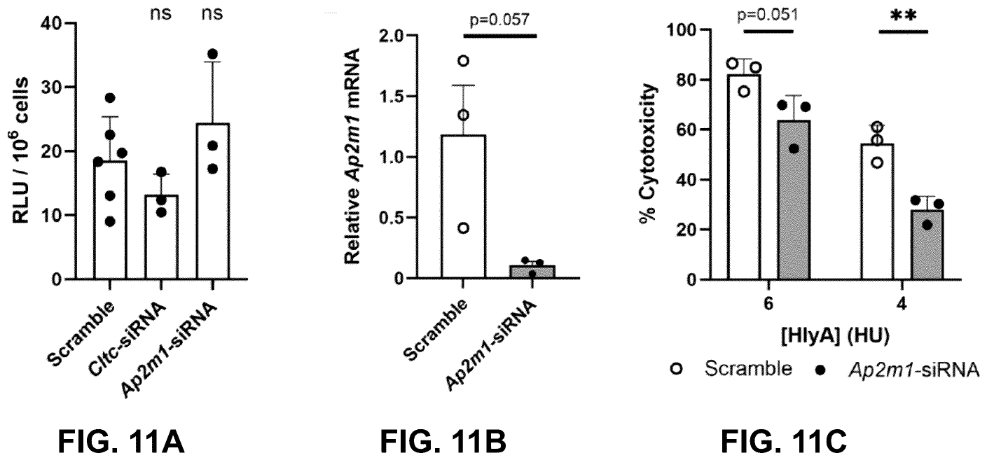

To validate a requirement for clathrin-mediated endocytosis in HlyA-mediated cytotoxicity to renal epithelial cells, pharmacological and genetic approaches were used. Dynamin-2, essential for scission of the invaginated membrane and separation of the endocytic vesicle, is inhibited by Dynasore and its derivatives, including Dyngo-4A. Pretreatment of IMCD-3 cells with Dyngo-4A before exposure to HlyA-CM reduced cytotoxicity in a dose-dependent manner, as compared to the vehicle (DMSO) control (FIG. 2D). The known on-target effects of Dyngo-4A were confirmed, as transferrin uptake was inhibited but total membrane cholesterol was unchanged (FIG. 10). Pretreatment with Dyngo-4A also abolished HlyA-mediated cytotoxicity of primary renal epithelial cells from C57BL/6 mice (FIG. 2E). To exclude off-target effects of the dynamin inhibitor, targeted downregulation of selected genes identified in the CRISPR screen with specific roles in clathrin-mediated endocytosis was also conducted. siRNA-mediated downregulation of Cltc (clathrin heavy chain) (FIG. 2F) reduced the susceptibility of IMCD-3 cells to HlyA-mediated cell death, compared to the scramble siRNA control (FIG. 2G). Analogous results were seen with siRNA-mediated downregulation of Ap2m1 (AP-2 complex, p subunit; FIG. 11).

### Internalized HlyA Permeabilizes Lysosomes and Initiates Necrotic Cell Death.

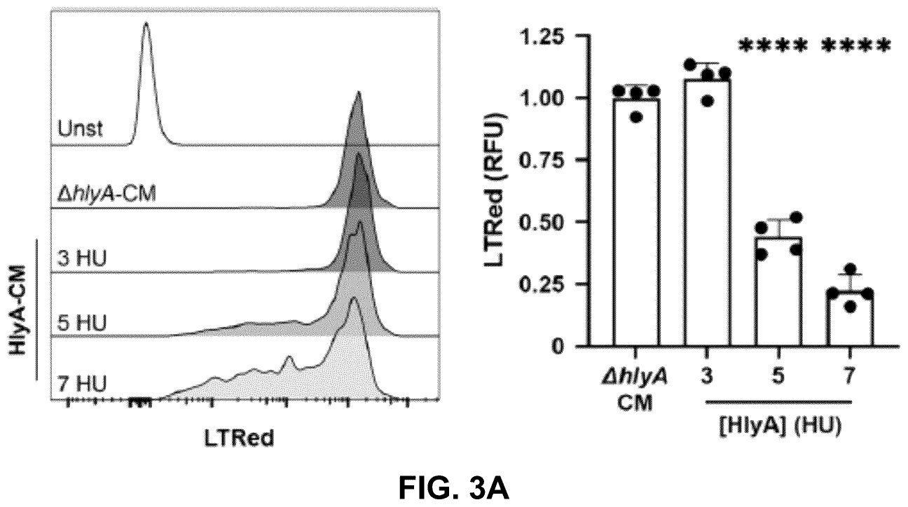

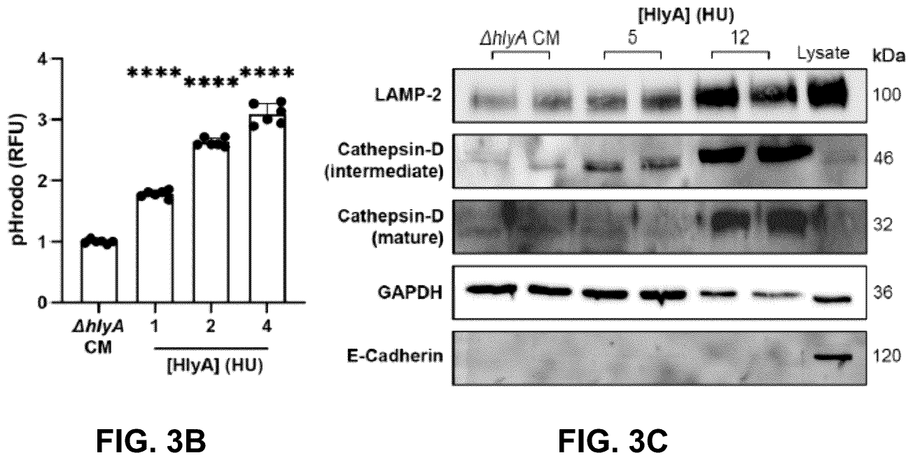

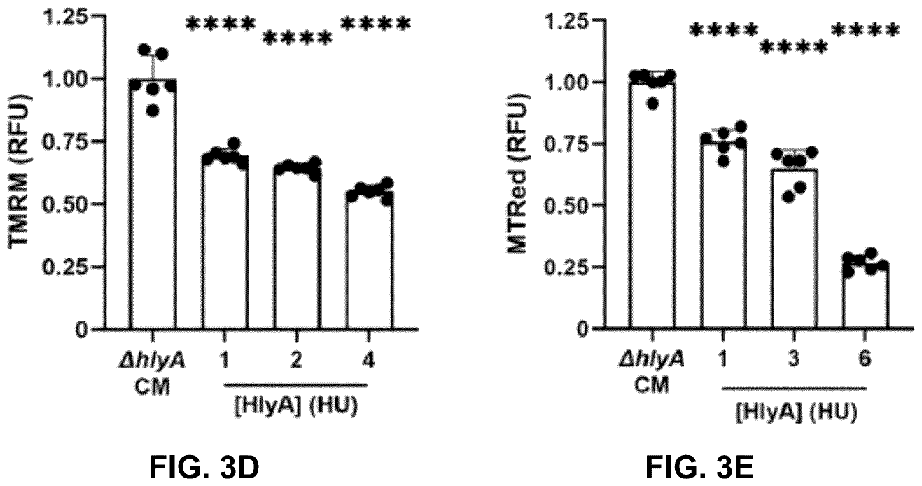

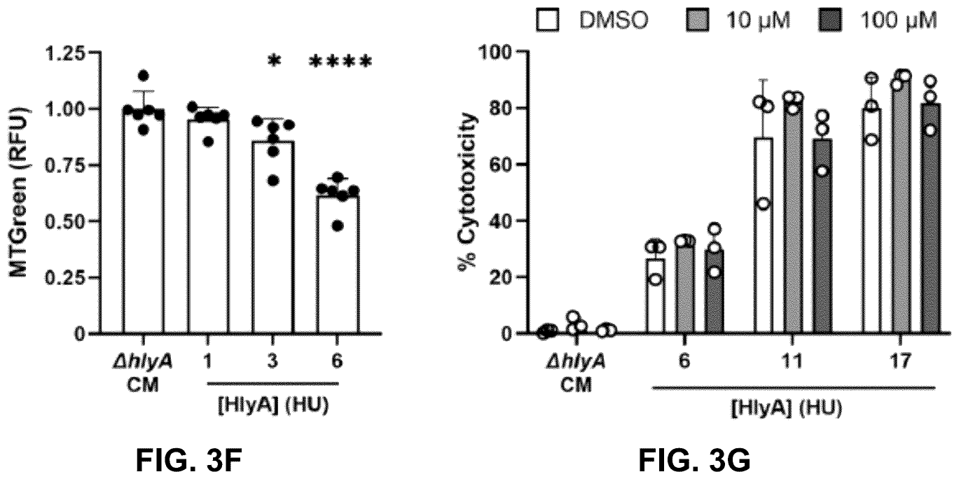

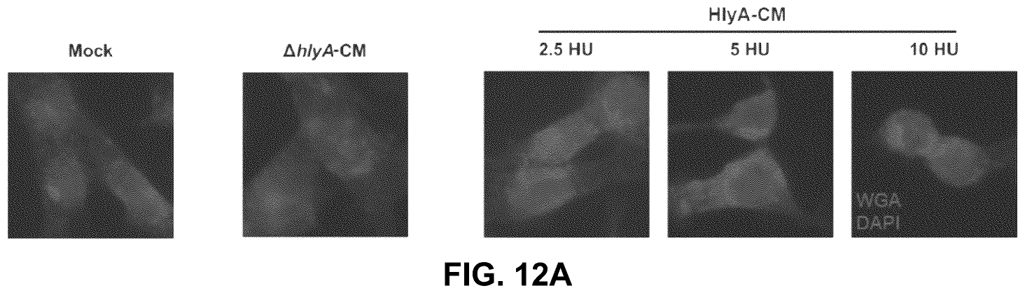

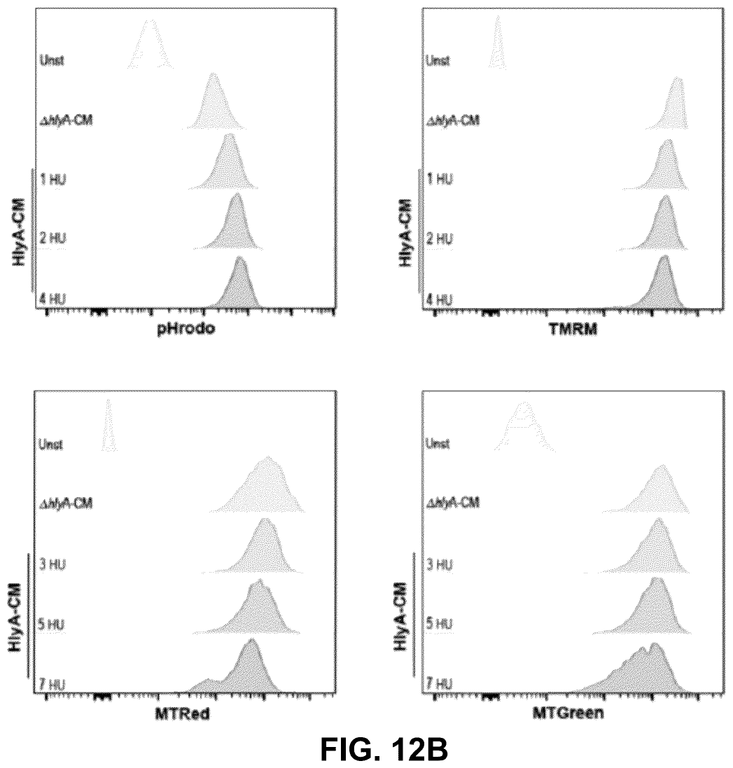

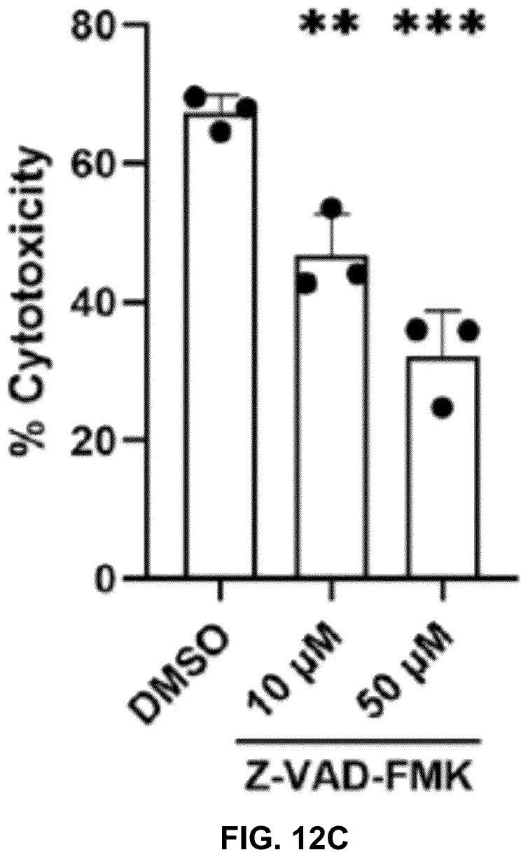

The genetic data suggested that HlyA internalization into epithelial cells is essential for its cytotoxic function, which contrasts with the hypothesis that HlyA lyses cell through pore formation in the plasma membrane. It was speculated that endocytosed HlyA might porate lysosomes, enabling the release of lysosomal contents and ultimately necrosis. IMCD-3 cells treated with HlyA-CM and examined by immunofluorescence microscopy exhibited cellular rounding and nuclear condensation in a dose-dependent manner, whereas these changes were not seen in cells exposed to ΔhlyA-CM (FIG. 12A). In contrast to cells treated with ΔhlyA-CM, those exposed to HlyA-CM after loading with LysoTracker Red dye exhibited a dose-dependent decrease in fluorescence, signifying lysosomal membrane permeabilization (FIG. 3A). HlyA-CM-exposed cells also exhibited a dose-dependent increase in pHrodo Green signal, reflecting cytoplasmic acidification, whereas cells exposed to ΔhlyA-CM did not show this change (FIG. 3B and FIG. 12B). Cellular fractionation was performed to assess for the presence of lysosomal proteins in the cytoplasm of HlyA-CM-treated IMCD-3 cells. Immunoblotting demonstrated a dose-dependent increase in the lysosomal membrane protein LAMP-2 within the cytoplasm, compared with ΔhlyA-CM-treated cells (FIG. 3C). Within endolysosomal vesicles, multiple classes of proteases, including cathepsins, aid in the recycling or elimination of endocytosed proteins. Immunoblotting demonstrated a dose-dependent increase in the 46-kDa intermediate form of cathepsin D and the 32-kDa mature form of cathepsin D in the cytoplasmic fraction of cells exposed to HlyA-CM (FIG. 3C). Cathepsins and other lysosomal proteases in an acidified cytoplasm can cause degradation of mitochondrial membrane proteins, with ensuing mitochondrial dysfunction and cytosolic oxidation. In a recent study, HlyA diminished the mitochondrial membrane potential (Δψ_{M}) and stimulated mitochondrial production of reactive oxygen species (ROS) in THP-1 macrophage-like cells. Exposure to HlyA-CM for 30 min in IMCD-3 cells loaded with the Δψ_{M} indicator dye tetramethylrhodamine (TMRM) resulted in decreased TMRM fluorescence, compared with ΔhlyA-CM-treated cells (FIGS. 3D and 12B). Loss of mitochondrial membrane potential and mitochondrial mass was confirmed by diminished MitoTracker Red (FIG. 3E) and MitoTracker Green (FIG. 3F) signals in HlyA-CM-exposed cells (FIG. 12B). Mitochondrial dysfunction can be part of several cell death pathways, and HlyA is reported to cause apoptotic or inflammatory cell death in various cell types. Here, pretreatment of IMCD-3 cells with the pancaspase inhibitor Z-VAD-FMK had no impact on HlyA-mediated cell death (FIG. 3G), while Z-VAD-FMK did inhibit staurosporine-induced apoptosis, as expected (FIG. 12C). These data are consistent with a model in which HlyA-induced lysosomal permeabilization leads to mitochondrial failure and cellular necrosis.

### LDLR Expression Facilitates HlyA Toxicity to Renal Epithelium

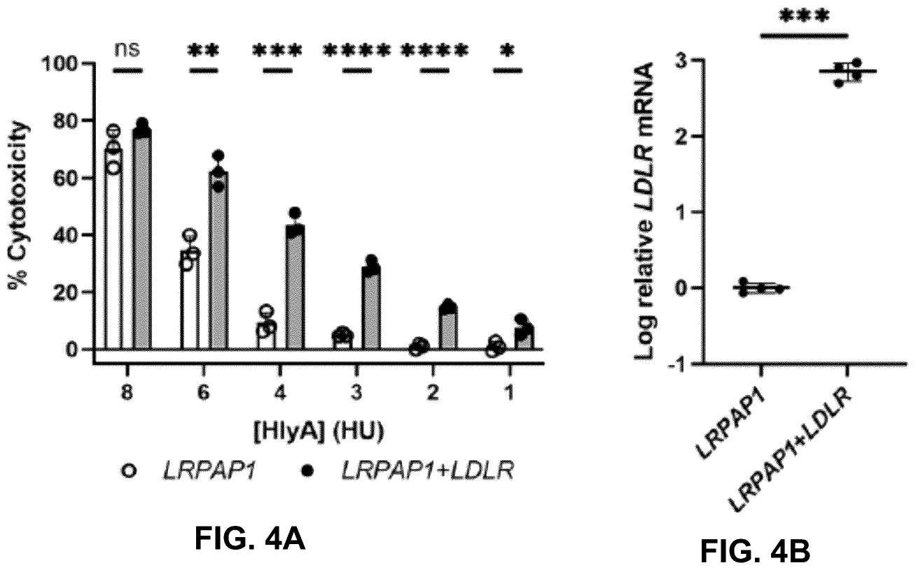

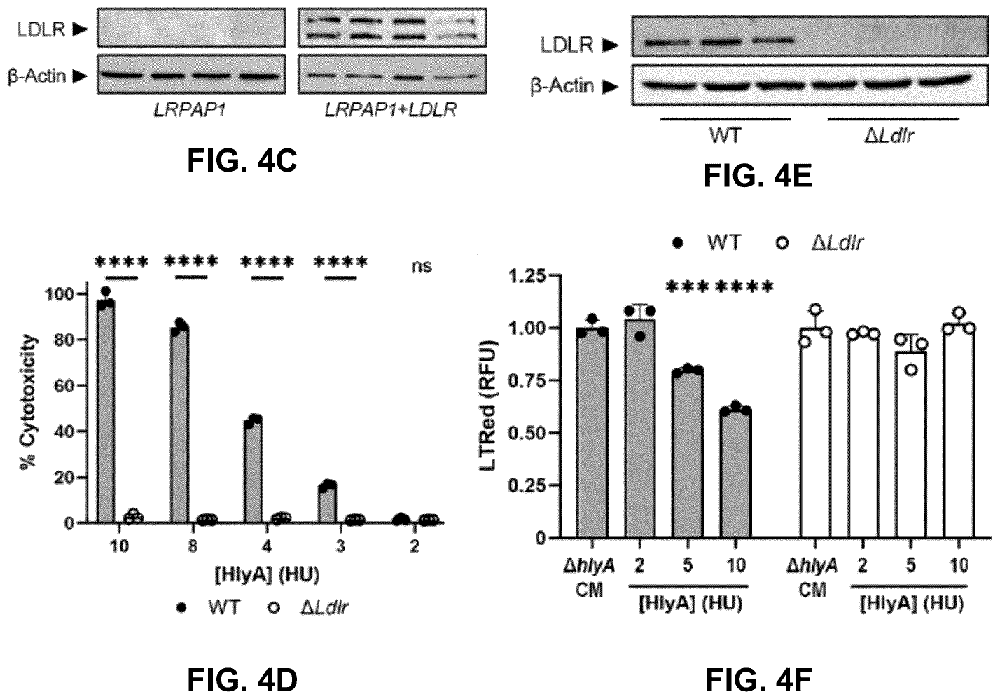

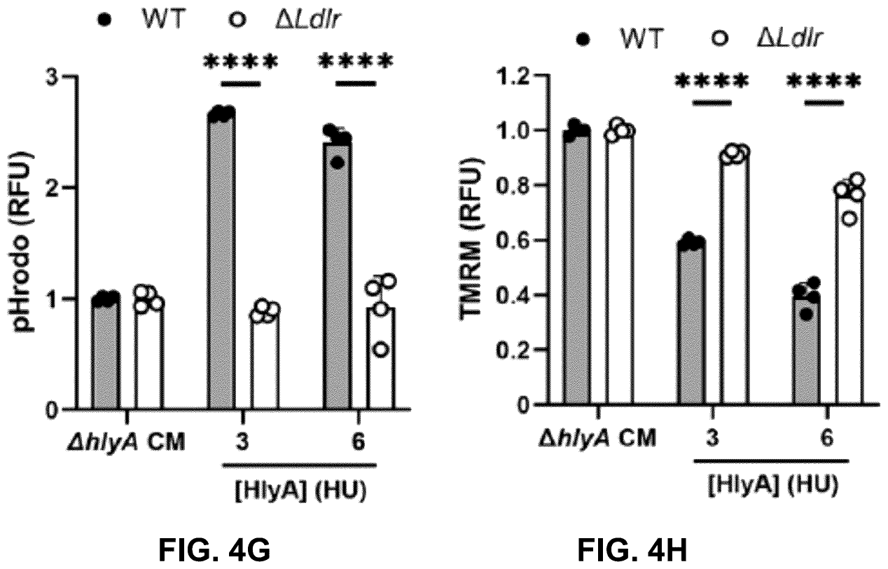

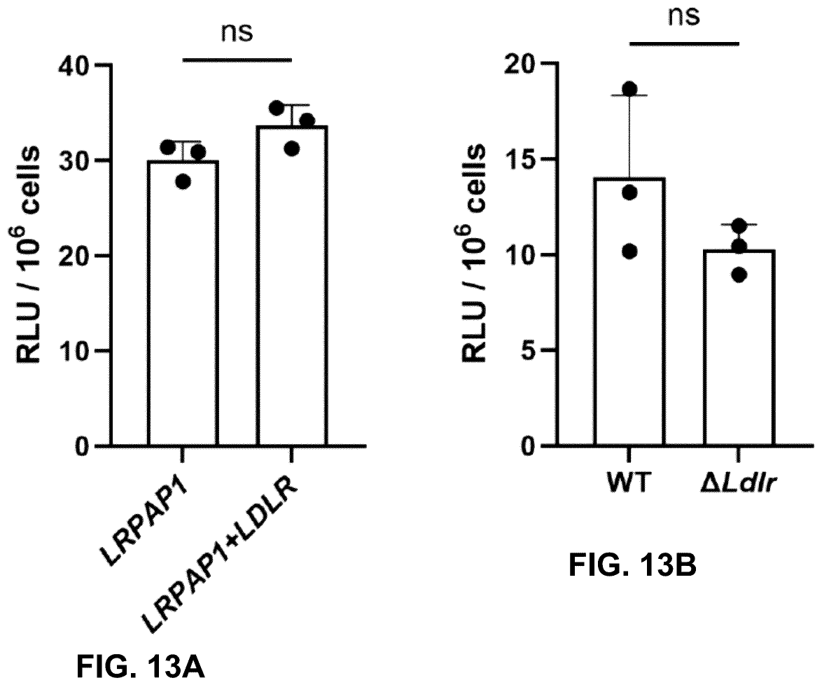

IMCD-3 cells with disruption of Ldlr were enriched in the CRISPR-Cas9 screen, suggesting that expression of functional LDLR was required for HlyA-mediated cytotoxicity. Mammalian LDLR is a surface-exposed plasma membrane receptor for the low-density lipoprotein (LDL) particle, which has key roles in cholesterol homeostasis and the development of atherosclerosis. LDLR binds the ApoB protein component of the LDL particle, triggering clathrin-dependent endocytosis. LDLR has also been co-opted as a cellular receptor for certain viruses and Clostridioides difficile toxin A (TcdA) entry into cells. HEK293T cells express little to no LDLR and are insensitive to HlyA-mediated lysis (FIG. 8B). Cotransfection of HEK293T cells with human LDLR and the gene (LRPAP1) encoding the required chaperone RAP 24 h prior to HlyA-CM treatment conferred HlyA-CM sensitivity, compared with cells transfected with only LRPAP1 (FIG. 4A). Expression of LDLR was confirmed by qRT-PCR and immunoblotting (FIGS. 4B and C). Of note, membrane cholesterol content among these cell populations and conditions was unchanged (FIG. 13A), excluding cholesterol alterations as a primary explanation for the findings.

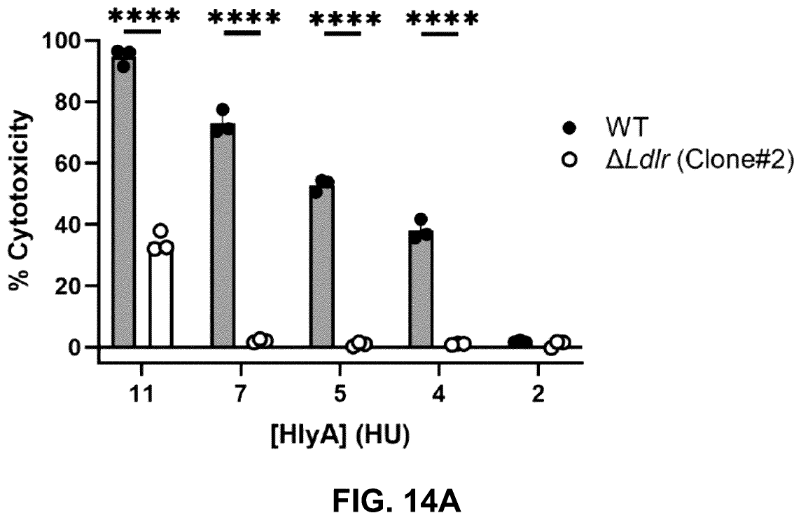

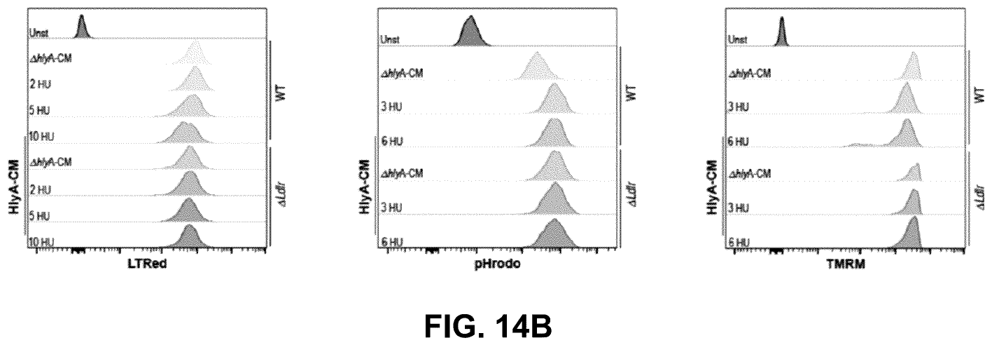

In an orthogonal approach, targeted CRISPR-Cas9 editing of Ldlr was performed in IMCD-3 cells. ΔLdir IMCD-3 cells resisted HlyA-mediated killing over all HlyA-CM concentrations tested, whereas wild-type IMCD-3 cells were susceptible to HlyA-dependent cytolysis (FIG. 4D). It was confirmed that Ldlr protein expression was absent in CRISPR-modified cells (FIG. 4E), with no impact on membrane cholesterol levels (FIG. 13B). The HlyA resistance phenotype was confirmed in a second, independently generated IMCD-3 ΔLdir clone to exclude off-target gene editing effects (FIG. 14A). IMCD-3 ΔLdlr cells also did not exhibit the phenotypes of lysosomal damage, cytoplasmic acidification, or loss of mitochondrial membrane potential (FIGS. 4F-H and 14B) that were seen in WT cells upon exposure to HlyA-CM. Taken together, these data show that LDLR expression in human or mouse epithelial cells promotes HlyA-mediated cytotoxicity.

Inhibition of LDLR-HlyA Interaction Protects Renal Epithelium from Injury.

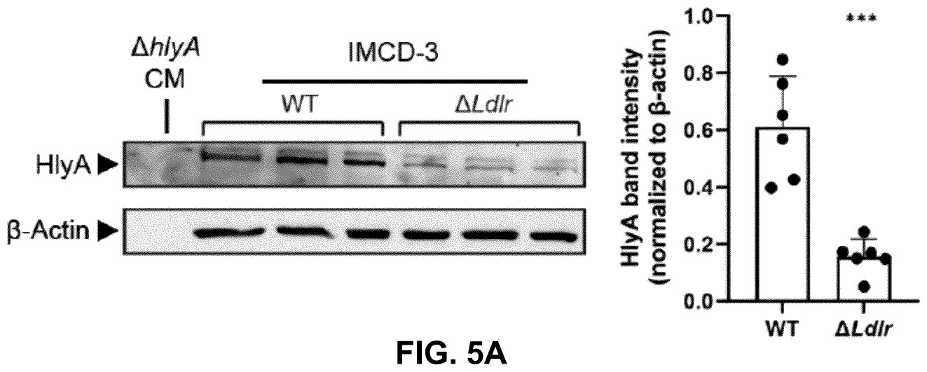

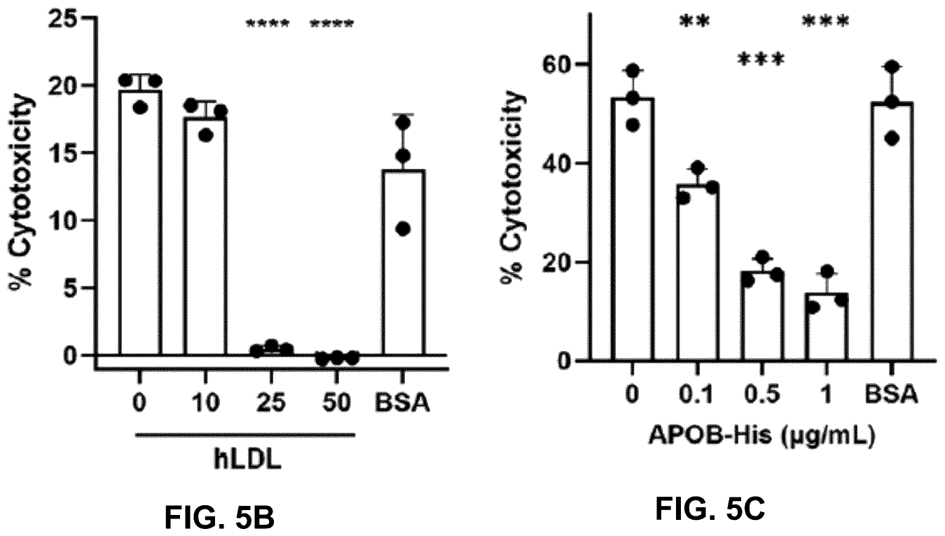

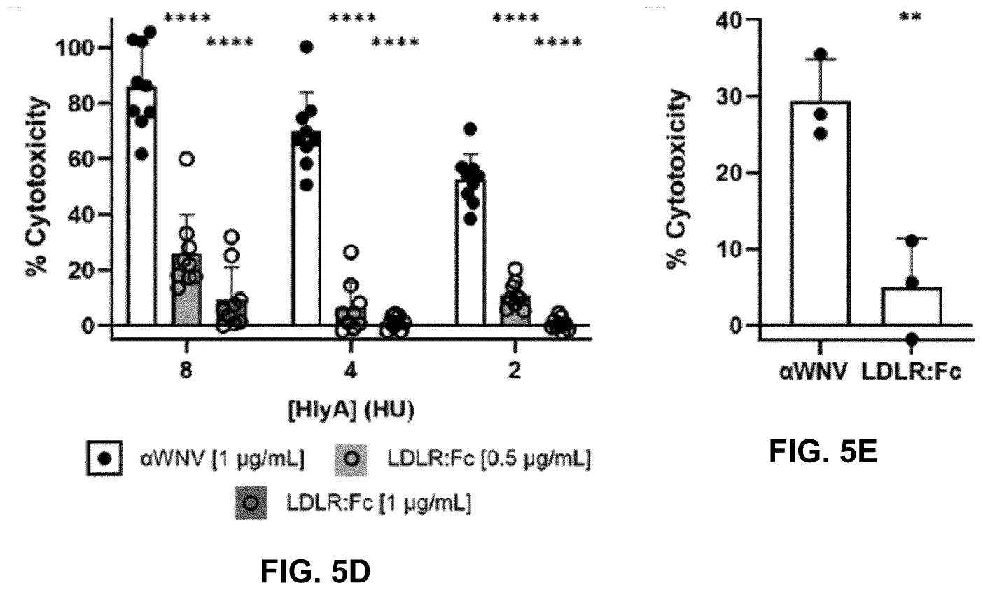

To determine whether HlyA binding to IMCD-3 cells depends on LDLR expression, whole-cell binding of HlyA was first studied using WT IMCD-3 or ΔLdlr cells that were pretreated with Dyngo-4A to block CME (and therefore cytotoxicity) before HlyA-CM exposure. Supporting a function for LDLR as a receptor for HlyA, more HlyA was found bound to WT compared to ΔLdlr IMCD-3 cells by immunoblotting cell lysates with an antibody recognizing HlyA (FIG. 5A). Furthermore, when physiologically relevant doses of purified human LDL (the canonical LDLR ligand) or a human APOB peptide (containing site B, the LDLR-binding moiety within LDL particles) were supplemented into serum-free HlyA-CM, dose-dependent reduction in cytotoxicity toward WT IMCD-3 cells was observed, whereas supplementation with bovine serum albumin (as control) was not protective (FIGS. 5B and C). Finally, emerging knowledge of LDLR-mediated viral entry has guided the development of protein-based molecules capable of protecting from infection with certain viruses. Accordingly, whether addition of a recombinant fusion protein containing the human LDLR ectodomain (LDLR-Fc) could function as a decoy receptor to protect renal epithelial cells from HlyA action was tested. Indeed, LDLR-Fc dose-dependently inhibited HlyA-induced toxicity toward IMCD-3 cells (FIG. 5D) and primary renal epithelial cells (FIG. 5E). Together, these results indicate that direct interaction between HlyA and LDLR precedes toxin internalization and cellular toxicity.

### Materials and Methods

### Bacterial Culture

UPEC strains utilized in this study were derived from the clinical isolate UT189. For murine UTI and for preparation of HlyA conditioned medium (CM; see below), bacteria were grown statically at 37° C. for 18 h in Luria-Bertani (LB) broth.

Genetic Manipulation of UT189 hlyA Expression

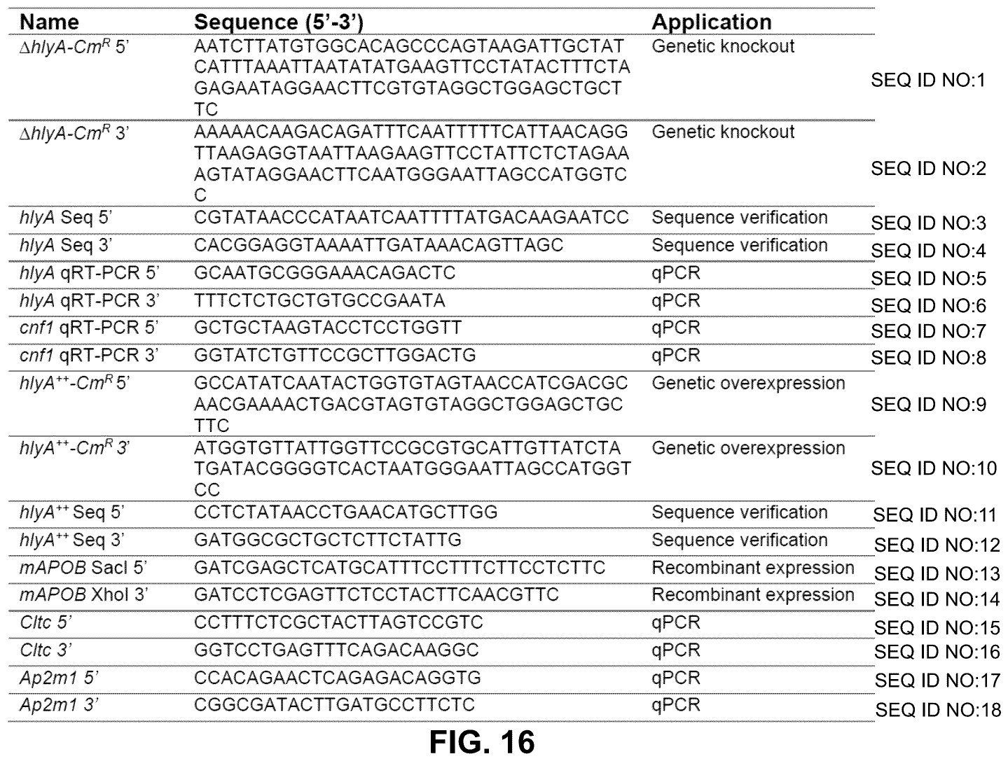

Targeted genetic deletion and chromosomal mediated overexpression of hlyA were achieved via the lambda red recombinase system. Oligonucleotides used are listed in FIG. 16 (SEQ ID NO: 1-SEQ ID NO: 18) and included in the Sequence Listing. For deletion of hlyA, a chloramphenicol resistance cassette (CmR) was amplified by PCR using primers comprising FLP recombinase sequences and regions complementary to those directly up- and downstream of the hlyA gene. This CmR linear fragment was transformed into UT189 carrying a plasmid (pKM208) expressing the lambda red recombinase. CmR clones were next transformed with the FLP expression plasmid pCP20 to effect excision of the CmR gene. To account for potential off-target effects, two independently derived isolates (ΔhlyA:1, ΔhlyA:2) were generated and tested. To induce stable HlyA overexpression in UT189, the CmR cassette was cloned directly upstream of the hlyCABD regulatory leader sequence. All clones were verified by sequencing, hlyA expression was confirmed by qPCR, and hemolytic activity was verified by plating on 5% sheep blood agar.

### Preparation of Conditioned Medium

CM were prepared daily and immediately prior to use in assays. As a result, modest daily variation in HlyA concentration (as measured by quantitative hemolysis; see below) was observed. Preparation of HlyA-CM utilized UT189 ΔhlyA:1 transformed with the HlyA expression plasmid pSF4000. Briefly, 3×10⁷ colony-forming units (CFU)/mL of ΔhlyA:1/pSF4000 or ΔhlyA:1 from static overnight LB broth culture was inoculated into complete or serum-free DMEM:F12 medium (as indicated) and incubated statically at 37° C. for 1 h. Bacteria were removed via centrifugation and passage through a 0.22-μm sterilizing filter. Multiple dilutions of HlyA-CM (in DMEM:F12) were used in the in vitro assays described below.

### HlyA Activity Quantification

Concurrent with use of daily HlyA-CM preparations in cytotoxicity studies (see below), toxin activity was quantified using a hemolysis assay with the following alterations. Prewashed rabbit RBCs were added to dilutions of HlyA-CM to a final concentration of 5% (v/v) in 500 μL, and the mixtures were incubated at 37° C. with gentle agitation for 1 h. Triton X-100 (0.1%) served as positive control. After centrifugation, the optical density of supernatants (540 nm) was quantified on a Tecan M-Plex microplate reader. One hemolytic unit (HU) was defined as the amount of toxin required to induce 50% lysis of RBCs, as has been classically defined. Linear regression was performed to quantify the activity (HU) present within each HlyA-CM preparation and dilution.

### Murine UTI

Female C57BL/6 mice (Envigo) were exposed to androgen, with 150 mg/kg testosterone cypionate (Depo-testosterone, Pfizer) via weekly intramuscular injection beginning at 5 wk and continuing until they were killed; control mice received similar injections of vehicle (cottonseed oil). Overnight UPEC cultures were centrifuged and resuspended to ˜5×10⁸ CFU/mL in PBS. At 8 to 9 wk, mice were inoculated via transurethral catheterization with ˜2.5×10⁷ CFU of the indicated UPEC strain. At the listed time points, mice were euthanized and organs harvested for downstream analyses. Bacterial loads were determined by serial dilution and plating of whole organ homogenates on LB agar. Hematoxylin and eosin (H&E)-stained sections were prepared by the Washington University Anatomic and Molecular Pathology core laboratory. Single sections were examined for signs of acute tubular injury and scored in 10% increments over the entire cortex by a blinded board-certified pathologist (J.P.G.). For Kim-1 immunohistochemistry (IHC), the left kidney was bisected longitudinally prior to fixation and paraffinization. 8-μm sections were mounted on glass slides. Antigen retrieval was performed by boiling in 10 mM sodium citrate. Sections were probed with goat anti-Kim1 (R&D Systems AF1817, 1:250) before incubation with secondary biotinylated mouse anti-goat IgG (1:250) (Invitrogen 31730, 1:250) followed by streptavidin-HRP (Thermo Scientific N504, 1:250).

Sections were treated with DAB substrate (Pierce) and dehydrated prior to mounting with glass coverslips. Kim-1-stained slides were examined and scored as percentage of positive tubules (defined as tubular brush border staining) in the most involved region of tissue (percent positive tubules per total tubules in 400× region). For Kim-1 quantification in serum, blood was collected 14 dpi via cardiac puncture and serum separated by centrifugation. Kim-1 ELISA (R&D Systems) was performed according to the manufacturer's instructions.

### Cytotoxicity Assays

To quantify HlyA-induced toxicity in vitro, epithelial cells were seeded into 96- or 24-well plates 24 h prior to HlyA-CM challenge. On the day of each experiment, medium was aspirated, and cells washed twice with sterile PBS prior to addition of HlyA-CM dilutions or control medium. Cells were incubated at 37° C. for 2 h, and then supernatants were removed and analyzed for lactate dehydrogenase (LDH) content using a commercial kit (Promega) per the manufacturer's instructions. Triton X-100 (0.1%) treatment served as a control representing maximal cell death (LDH release). For experiments with endocytosis inhibition, Dyngo-4A (Sigma 324413; in 0.1% DMSO) was incubated at the specified concentrations with cells in serum-free DMEM:F12, beginning 1 h prior to and maintained during HlyA-CM challenge; 0.1% DMSO was included as vehicle control. In other experiments, recombinant LDLR ectodomain fusion peptide (LDLR-Fc; ACRO Biosystems), an unrelated control antibody (anti-West Nile virus hE16), purified human low-density lipoprotein (LDL) cholesterol (ThermoFisher #L3486), human APOB-His (LS Bio #LS-G14619), or bovine serum albumin (BSA, as control; ThermoFisher #BP9703) was added as specified, concurrent with HlyA-CM challenge. To ascertain the participation of caspases in HlyA-mediated cell death, cells were incubated with the pancaspase inhibitor Z-VAD-FMK for 1 h prior to treatment with CM or staurosporine (1 μM). For all in vitro assays reported, data shown are representative of at least three independent experiments unless otherwise specified herein.

### CRISPR-Cas9 Genetic Screen

The generation of a library of CRISPR-Cas9-modified murine IMCD-3 cells transduced with the Brie sgRNA library was produced, representing roughly ˜20,000 mouse genes (4 sgRNAs per gene). Guide coverage of a freshly expanded aliquot of these cells was confirmed by sequencing. 5×10⁶ cells were seeded into 175-cm² flasks in complete DMEM:F12 containing puromycin and blasticidin; 24 h after seeding, monolayers were exposed to ˜10 to 12 HU of HlyA-CM at 37° C. for 2 h, visually achieving ˜60 to 80% cell death in the first round. The flask was gently washed with Dulbecco's PBS (DPBS), medium was replaced with fresh complete DMEM:F12, and surviving cells were allowed to recover for 48 h. After recovery, cells were split 1:2 into new flasks; one flask proceeded to the next round of toxin exposure, while the other was saved for genomic DNA extraction. Sequencing was performed by the Genome Technology Access Center (GTAC@MGI) at Washington University, using the MAGeCK package (version 0.5.9) (73) to generate read count tables for each sample and comparing conditions using the RRA algorithm. Candidate genes considered significant were those exhibiting average log fold change >2 and false discovery rate <1%.

### CRISPR-Mediated Ldlr Disruption

Ldlr KO lines were created by the Genome Engineering & Stem Cell Center (GESC@MGI) at Washington University. Briefly, synthetic gRNA targeting the sequence 5′-CAGGAATGCATCGGCTGACANGG-3′ (SEQ ID NO: 19) cells. Transfected cells were then single-cell sorted into 96-well plates, and clones were sequenced to identify the indels desired for gene disruption. Two independent ΔLdir clones were generated and studied.

### Transferrin Uptake

Cells were seeded at a density of 1.5×10⁵ cells per well in 24-well plates. The following day, medium was removed, and wells were washed with PBS before adding warm serum-free medium. Plates were incubated at 37° C. with 5% CO₂ for 30 min. Medium was then replaced with warm serum-free medium containing Dyngo-4A or DMSO (negative control) and incubated for an additional 30 min at 37° C. with 5% CO₂. Subsequently, fresh cold serum-free medium containing 25 μg/mL of AlexaFluor 488-labeled transferrin (JacksonImmuno #015-540-050) and either Dyngo-4A or DMSO (control) were added, and plates were incubated on ice for 10 min in the dark. Plates were transferred to a 37° C. incubator for the indicated time intervals, after which they were promptly returned to ice. Medium was removed, and wells were washed twice with ice-cold PBS, twice with 500 μL ice-cold acid wash buffer, and twice more with ice-cold PBS. Next, 200 μL of 0.25% trypsin was added, and cells were incubated at room temperature for 3 min; 1 mL of PBS was added, and cells were transferred to FACS tubes. Cells were centrifuged and washed twice with PBS, then filtered into new FACS tubes; just prior to analysis, 200 ng of propidium iodide (PI) was added. Cells were analyzed on a Cytek Aurora instrument, and data were analyzed with FlowJo software.

### Lysosomal and Mitochondrial Assays

IMCD-3 cells (1.5×10⁵ per well) were seeded in 24-well plates with complete DMEM-F12 for 24 h. Cells were washed with PBS, then incubated with one of the following: 50 nM LysoTracker Red (ThermoFisher), pHrodo Green (ThermoFisher), MitoTracker Red, or MitoTracker Green, in serum-free DMEM:F12 for 30 min at 37° C. Cells were washed twice with PBS to remove excess dye and then exposed to HlyA-CM for 30 min at 37° C. Cells were liberated with trypsin as described above and washed twice with PBS in FACS tubes; PI was added immediately prior to analysis.

### Membrane Cholesterol Content

IMCD-3 monolayers grown to 80 to 90% confluency in 6-well plates were serum-starved for 30 min prior to incubation with Dyngo-4A or DMSO control in serum-free DMEM:F12. HEK293T cells transfected with LRPAP1 or LRPAP1+LDLR were grown to 80 to 90% confluency in six-well plates. In both cases, cells were liberated with 0.25% trypsin and washed with PBS prior to analysis with a commercial assay kit (Promega #J3190) according to the manufacturer's instructions.

### Protein Extraction, Cellular Fractionation, and Immunoblotting

To measure LDLR expression, cells in 24-well plates were washed 3 times with 0.5 mL of cold PBS, then lysed with 0.5 mL of 1×RIPA buffer+Halt Protease Inhibitor Cocktail (ThermoFisher). For HlyA whole-cell binding analysis, IMCD-3 cells grown to 80 to 90% confluency in T25 flasks were lysed with 0.2 mL 1×RIPA buffer+Halt Protease Inhibitor Cocktail and centrifuged at 16,000×g for 5 min at 4° C. to remove debris. For isolation of cytoplasmic fractions, IMCD-3 cells were grown to 80 to 90% confluency in T75 flasks. Cells were exposed to indicated amounts of HlyA-CM or ΔhlyA-CM for 30 min at 37° C. Monolayers were washed with cold PBS, and cytoplasmic fractions were extracted with a commercial kit (ThermoFisher #78840). Protein concentration in all cellular extracts were quantified by BCA assay (Pierce).

For immunoblotting of cell lysates, 40 μg of lysate was loaded per well and separated on polyacrylamide gels. After transfer to nitrocellulose membranes, lysates were probed overnight with rabbit anti-LDLR (Invitrogen #MA5-32075, 1:1,000), Rabbit anti-β-actin (Cell Signaling Tech #4967, 1:20,000), or mouse anti-HlyA (74) (H10, 1:2,000). Blots were incubated with the appropriate secondary antibodies for 1 h: donkey anti-rabbit-HRP (Cytiva #NA934V, 1:5,000) or sheep anti-mouse-HRP (Cytiva #NA931V, 1:5,000). Blots were visualized using western ECL substrate (Bio-Rad #1705060) and imaged on a Bio-Rad ChemiDoc MP instrument. For HlyA whole-cell binding analysis, HlyA band intensities were quantified via ImageJ (Fiji 2) and normalized to β-actin.

### Transfection

Expression vectors for LRPAP1 and LDLR were used. Plasmid DNA was extracted from 250-mL LB broth cultures using the Qiagen Plasmid Maxi Kit (Qiagen #12163). HEK293T cells grown to ˜75% confluency in a T75 flask were transfected with 30 μL of Lipofectamine LTX DNA Transfection Reagent, 10 μL of PLUS Reagent (Life Technologies #15338-100), and 5 μg of LRPAP1 plasmid DNA with or without 10 μg of LDLR plasmid DNA. Approximately 18 h later, cells were plated (2×10⁵ cells per well in 24-well plates); 24 h after seeding, cells were exposed to HlyA-CM or harvested for LDLR immunoblotting.

siRNA Treatment

IMCD-3 cells were seeded in 24-well plates (˜10⁵ cells/well); 18 h later, cells were transfected with 50 nM of siRNA and 1 μL of Lipofectamine 2000 (ThermoFisher #11668027) per well following the manufacturer's instructions. One day posttransfection, cells were used for cytotoxicity assays or RNA extraction. The fluorescent reporter TYE563 and housekeeping gene Hprt were used as positive controls to verify transfection, and scramble siRNA was used as control for Cltc and Ap2m1 siRNA knockdown.

RNA Extraction and qPCR

Cells were grown to ˜80 to 90% confluency in 24-well plates, medium was removed and cells washed with PBS. RNA was isolated with RNA STAT-60 (Tel-Test Inc.) and cDNA synthesized (BioRad iScript) per respective manufacturer's protocols. qPCR was performed using SYBR Green Supermix (BioRad). Each reaction contained 10 μL of SYBR Green master mix, 20 ng cDNA, and 0.5 μM forward and reverse primers (FIG. 16, SEQ ID NO: 1-SEQ ID NO: 18) in a final volume of 20 μL.

### Statistical Analysis

Pairwise comparisons of in vivo bacterial titers and renal pathology were made using the nonparametric Mann-Whitney U test. Other pairwise comparisons were made with unpaired t tests. Multiple comparisons were made using ANOVA with Tukey post hoc or Dunnett multiple comparison tests as indicated. P values≤0.05 were considered significant. All numerical data were analyzed using GraphPad Prism 10.4.

### Discussion

The UPEC α-hemolysin (HlyA) can exert cytotoxicity in a range of mammalian cell types. In Example 1, it is demonstrated that HlyA action on renal epithelium relies on clathrin-mediated endocytosis and LDLR expression, and that rapid cell death ensues from lysosomal permeabilization, cytoplasmic acidification, and resulting mitochondrial injury. This work reveals a distinct paradigm for the biological activity of HlyA, a prototypic member of the RTX family that has been studied for decades but whose mechanisms of action have been elusive.

Compared with other families of bacterial pore-forming toxins (PFTs), the RTX toxins—comprising over 1,000 members across a wide range of pathogenic species—have proven more enigmatic despite extensive study: Structural determinations have been difficult, diversity among their functional domains is broad, and even how the canonical acylation step enables toxin function is not understood. HlyA was initially isolated based on its capacity to lyse RBCs. The traditional view that HlyA kills cells via poration of the plasma membrane is based on biochemical and membrane-conductance observations with RBCs and artificial membranes. Similarly, studies of myeloid cell susceptibility to HlyA-mediated killing have been consistent with this mode of action. Nonetheless, typical purification and structure elucidation has been precluded by the instability and thermolability of the toxin, which was recognized some 60 y ago. As a result, prior work with HlyA (and other RTX toxins) has relied in part on harsh purification and reconstitution protocols that might affect toxin activity. In contrast, the use of conditioned medium from an overexpressing UPEC strain, prepared fresh daily, prioritizes the retention of HlyA's biological activity during experimentation. This toxin preparation strategy demonstrated that epithelial cells are more susceptible to HlyA than myeloid cells, consistent with one other report that used natively produced toxin against A498 renal epithelial cells. The apparent preference of HlyA for myeloid cells (particularly over RBCs) has been attributed to their expression of CD11a/CD18 (LFA-1). Most recently, screening of a mutant library in myeloid U937 cells exposed to HlyA identified CD18, the integrin P2 subunit, as the most prominent hit. CD18 was shown to bind HlyA and a related RTX toxin (Aggregatibacter actinomycetemcomitans RtxA), and siRNA-mediated downregulation of CD18 protected U937 cells from HlyA action. As higher HlyA toxicity to renal epithelium was observed, and as epithelial cells do not express CD18, alternative cellular mediators of HlyA action were sought using a genetic screen. A requirement for LDLR expression and clathrin-mediated endocytosis for HlyA activity in epithelial cells was identified. The present findings do not refute earlier proposed mechanisms of HlyA action but rather expand the understanding of the activities of this and potentially other RTX family members.

Clathrin-mediated endocytosis is a common form of receptor-dependent ligand internalization in mammalian cells. Clathrin-mediated endocytosis and LDLR family members have been implicated in the pathogenic activity of other microbial toxins, though from families other than RTX, including the large Clostridium toxin (LCT) family. For example, activity of C. difficile exotoxin TcdA is reduced in epithelial cells lacking LDLR, though direct TcdA-LDLR interaction was not observed, suggesting LDLR might have a facilitating role after initial TcdA binding to surface glycosaminoglycans. Similarly, several LDLR family members (including LDLR, LRP1, and megalin) act via their ligand-binding domains to promote internalization of Clostridium novyi alpha-toxin (Tcna). LRP1 also serves as a receptor for the uniquely structured vacuolating cytotoxin (VacA) of Helicobacter pylori, the Pseudomonas aeruginosa exotoxin A (an ADP-ribosylating toxin), and the deaminase toxin of Pasteurella multocida. Anthrax toxin, an example of the A-B toxin class, gains entry to target cells via the LDLR family member LRP6. Importantly, while these examples illustrate how LDLR family members participate in microbial toxin internalization, they belong to toxin classes—such as enzymatic or A-B toxins—that typically require entry into the cytoplasm to exert their effects. In contrast, the findings with HlyA in Example 1 represent the unexpected demonstration of a PFT requiring internalization via LDLR-mediated, clathrin-dependent endocytosis in order to exert its cytotoxic activity. As noted earlier, LDLR family members are also co-opted for cellular entry by an array of viruses, including encephalitic alphaviruses, vesicular stomatitis virus, and hepatitis B virus. The screen identified LDLR itself as a primary HlyA receptor on renal epithelial cells, but it is anticipated that other LDLR family members might specifically or redundantly facilitate HlyA toxicity, given their sequence and structural relatedness and based on their relative expression by various cell types. Of note, among the multiple means of LDLR inhibition utilized, ApoB and LDL, as natural ligands for LDLR, might additionally reduce HlyA toxicity by triggering internalization of the receptor.

Cholesterol in target membranes is essential to the stabilization and functionality of many PFTs, including HlyA and a separate toxin family in Gram-positive bacteria, the cholesterol-dependent cytolysins. It was verified that the results observed during perturbations of clathrin-mediated endocytosis and LDLR expression and the competitive inhibition studies were not due to altered plasma membrane cholesterol content, which might have imparted a nonspecific effect on HlyA activity. Another significant hit in the CRISPR screen was Npc1, which encodes a well-characterized lysosomal cholesterol transporter. Cholesterol may be important for lysosomal permeabilization, one of the first events following cellular internalization of HlyA. Lysosomal proteases were detected in the cytoplasm of HlyA-intoxicated cells, and the accompanying cytoplasmic acidification might promote the activity of these proteases in this compartment. This model aligns with an earlier study reporting that in bladder epithelial cells treated with sublytic concentrations of HlyA, cellular protease activity in the cytoplasmic compartment led to degradation of cytoskeletal proteins such as paxillin. HlyA pore formation in the lysosomal membrane is suspected. Inherent to their function, and unlike other intracellular organelles, the interior of lysosomes is significantly more concentrated than that of the surrounding cytoplasm. Recent works highlight lysosomal sensitivity to osmotic stress and the importance of lysosomal osmo-regulating proteins in maintaining the organelle's integrity. Upon HlyA-mediated poration, ion and solute flux would ultimately result in osmotic disruption of the lysosome. Moreover, at least two recent studies have demonstrated that lysosomal protease activity in the cytoplasm causes degradation of mitochondrial proteins and impairment of mitochondrial membrane potential and function, matching the sequence of events observed during the rapid HlyA-induced and caspase-independent death of renal epithelial cells. Finally, another recent paper posited that HlyA promotes persistence of UPEC within bladder epithelial cells; the mechanism involved disruption of microtubule organization, impaired recruitment of vacuolar ATPase recruitment to lysosomes, and resulting failure of lysosomes to acidify, thereby hampering killing of intracellular UPEC. Although this study focuses on the internalization of extracellularly produced HlyA (rather than HlyA secreted by internalized bacteria), it is suspected that HlyA-mediated disruption of lysosomal osmotic homeostasis underlies observations in both models.

In summary, the findings in Example 1 introduce a distinct mechanism of action for HlyA, and potentially for other members of the very broad family of RTX toxins. The identification of a requirement for endocytosis distinguishes HlyA from existing dogma that PFTs (including HlyA) act primarily by plasma membrane disruption. The specific effects of HlyA on a given target cell likely depend on receptor expression, other plasma membrane characteristics, and toxin dose, as well as experimental (e.g., purification) conditions. Therefore, HlyA reliance on LDLR-mediated endocytosis represents its primary mode of action, at least toward epithelial cells; meanwhile, plasma membrane poration by HlyA might require toxin concentrations higher than those in contexts such as UPEC infection of the kidney. Ongoing work includes further specification of HlyA trafficking after internalization, interrogation of other LDLR family members as potential mediators of HlyA toxicity in other cells, and efforts to elucidate structural features of the HlyA interaction with LDLR. The present work also illuminates possible anti-virulence therapeutic avenues to prevent tissue injury during UPEC pyelonephritis and other infections caused by HlyA-secreting E. coli.

The publication of Kuhn et al. entitled “LDL receptor-mediated endocytosis of Escherichia coli α-hemolysin mediates renal epithelial toxicity”, Proc Natl Acad Sci USA, June 2025, vol. 122(24), article no. e2505482122 is incorporated herein by reference in its entirety as support of Example 1.

### Example 2—HlyA-Mediated, CME-Dependent Toxicity to Myeloid and Endothelial Cells and its Relationship to Severity of Outcomes of E. coli Sepsis

Sepsis is an important cause of death in adults and children worldwide. In 2020, 11 million sepsis-related deaths were estimated, representing 20% of all global deaths. Escherichia coli is the leading Gram-negative etiology of bacteremia and sepsis. E. coli is classified into an array of pathotypes, several of which cause symptomatic intestinal infections; outside of the gut, so-called extraintestinal pathogenic E. coli (ExPEC) commonly cause urinary tract infections (UTI, where they are termed uropathogenic E. coli [UPEC]), peritonitis, sepsis and other life-threatening infections. Though common bacterial factors such as lipopolysaccharide (LPS) and host cytokine and cellular responses have been studied extensively in sepsis models, adjunctive therapeutics (beyond antibiotics) have not achieved clinical utility, likely because cellular and molecular mechanisms underlying sepsis vary by causative pathogen. E. coli, like many other bacterial pathogens, employ an array of virulence factors to establish infection, resist host immune defenses, and cause tissue damage and pathology. Among these is the secreted alpha-hemolysin (HlyA), a toxin recognized for decades as exerting cytotoxicity toward erythrocytes, myeloid cells, and some epithelial cells but whose functions have been challenging to decipher.

Though it has long been thought that HlyA acts by forming pores in the plasma membrane of target cells, recent work in models of pyelonephritis revealed a completely novel paradigm for its mechanism of action. Specifically, HlyA binds the low-density lipoprotein receptor (LDLR) and is internalized via clathrin-mediated endocytosis (CME), leading to lysosomal permeabilization, mitochondrial dysfunction, and rapid death of renal epithelial cells. Genetic abrogation or competitive inhibition of the HlyA-LDLR interaction is highly protective against HlyA-mediated epithelial cell death. This set of findings illuminates new avenues toward anti-virulence strategies for mitigation of host cell damage in E. coli infections. A murine model of E. coli sepsis was established, utilizing the UPEC strain CFT073, in which secretion of HlyA causes sharply higher morbidity and mortality. It is proposed that E. coli sepsis severity is driven by HlyA-mediated damage to myeloid cells (critical responders to bacterial infection) and to endothelial cells (ECs, key mediators of sepsis pathophysiology including vascular permeability and intravascular coagulation). Though a candidate HlyA receptor (the β2-integrin CD18, encoded by the Itgb2 gene) has been proposed on macrophages, preliminary data suggest that endocytosis of HlyA is necessary for its toxicity to these cells; meanwhile, minimal work has been done on HlyA intoxication of ECs.

There is a promising opportunity to define the molecular mechanisms of HlyA toxicity to these critical cell populations, and to illuminate how inhibition of key toxin-host cell interactions might mitigate sepsis severity. Example 2 addresses the severity and outcomes of E. coli sepsis as determined by HlyA-mediated, CME-dependent toxicity to myeloid and endothelial cells.

The goals of Example 2 are three-fold:

### Redefine the Mechanisms of HlyA Toxicity to Myeloid Cells.

Published studies using cultured macrophage-like (THP-1) or monocytic (U937) cells argue that HlyA utilizes CD18 as a receptor to enable pore formation in the plasma membrane of target cells. It is posited that in addition to (or instead of) this proposed mechanism, myeloid cells are intoxicated via endocytic internalization of HlyA, as observed in epithelial cells. Preliminary data confirm dose-dependent susceptibility of THP-1 and U937 cells to HlyA and newly implicate endocytosis as required for toxicity. Whether lack of LDLR and/or CD18 (achieved via siRNA or CRISPR-mediated deletion) or competitive inhibition (with LDLR ectodomain, the canonical LDLR ligand APOB, or LDLR- or CD18-blocking antibodies) protects these cells from HlyA-mediated death are tested. The pathway(s) of HlyA-mediated cell death in myeloid cells are characterized, measuring lysosomal permeability, mitochondrial dysfunction and cytokine production, and protection is tested by caspase inhibitors and other agents. Lastly, the mechanisms of HlyA toxicity toward human neutrophils isolated from healthy donors are defined.

### Define the Mechanisms of HlyA Toxicity to and Activation of Endothelial Cells.

HlyA action on ECs was the subject of only a few studies several decades ago, and no surface receptors for HlyA on ECs are known. Cultured ECs express LDLR (and other family members) and can express low levels of CD18, and are key drivers of cytokine signaling, neutrophil trafficking, and intravascular coagulation in sepsis. It has been shown in Example 1 that dose-dependent susceptibility of cultured human ECs to HlyA, and in this section, whether and how endocytosis of LDLR and/or CD18 mediate HlyA cytotoxicity to ECs are specified, using approaches discussed earlier. In addition, mechanisms by which HlyA exposure activates EC pro-coagulant and pro-inflammatory responses and induces endothelial permeability are interrogated, all important components of sepsis pathophysiology.

Target Mediators of HlyA Action on Key Cell Populations to Mitigate E. coli Sepsis In Vivo.

Preliminary data show that deletion of hlyA from CFT073 reduces morbidity and mortality in murine sepsis. The pathologic manifestations of sepsis are detailed in this model, including end-organ damage, vascular permeability, and disseminated intravascular coagulation (DIC), and discern their dependence on HlyA. Whether mice with conditional deletion of Ldlr and/or Itgb2 from myeloid cells (using LysM-Cre), specific myeloid populations (e.g., Mrp8-Cre), or endothelial cells (Tie2-Cre) are protected from mortality and morbidity is tested. Whether pharmacologic targeting of HlyA-LDLR and/or HlyA-CD18 interactions can mitigate the morbidity and mortality of CFT073 sepsis in wild-type mice is also tested.

### Significance and Premise

### Rigor of Prior Research on HlyA

The in vitro hemolytic activity of many E. coli strains was recognized ˜100 years ago, and there have been many studies of its toxicity toward various (mostly hematopoietic) cell types and potential mechanisms of action. Among the various pathotypes of E. coli, HlyA is carried primarily by UPEC strains, while a small minority of enteroaggregative E. coli strains also carry it. HlyA is encoded within the four-gene hlyCABD operon and has a molecular weight of ˜110 kDa. As a member of the repeats-in-toxin (RTX) family, mature HlyA contains a repeating nonapeptide sequence that binds Ca2+ ions, enhancing proper folding after secretion. The hlyC gene encodes an acyltransferase that post-translationally modifies newly synthesized HlyA, a modification necessary for extracellular Ca2+ binding and cytotoxic activity. The other gene products (HlyB and HlyD) facilitate HlyA delivery to the TolC apparatus for extracellular release (i.e., type I secretion). Due to its ability to lyse erythrocytes and myeloid cells, HlyA has been proposed to act by forming pores in the plasma membrane of target cells. Though one study asserted that HlyA appears to form small pores in artificial bilayered membranes, other studies suggest that HlyA forms an atypical pore that enlarges (in the erythrocyte membrane) with higher toxin concentration, or instead exerts detergent-like activity to disrupt one leaflet of a typical membrane. Purification of the toxin has been challenging, likely due to its amphipathic nature and its instability in solution and other milieux even after acylation and Ca2+ binding. As a result, no crystal or cryo-EM structure of the toxin has been obtained, and HlyA pores have not been observed in target cell plasma membranes by EM.

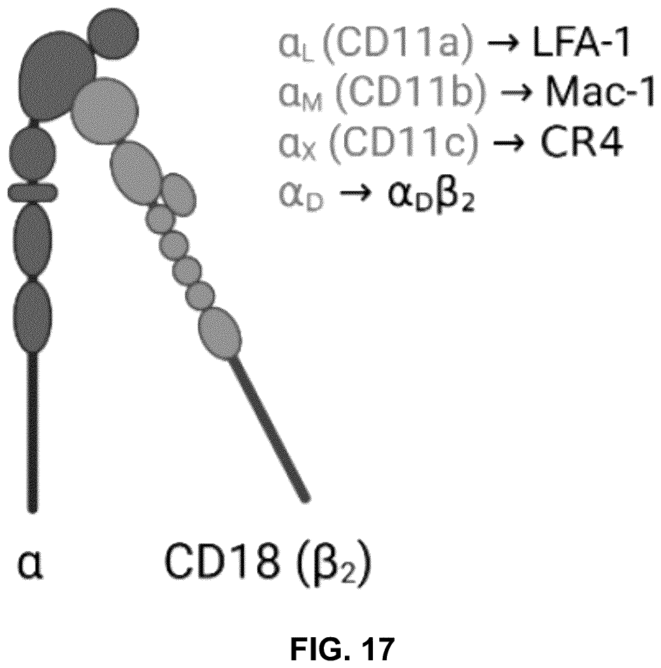

HlyA, like several other RTX toxins (such as Aggregatibacter LtxA), has been proposed to exert toxicity to myeloid cells via plasma membrane poration upon interaction with the β2-integrin CD18, which is expressed within heterodimers on the surfaces of leukocytes (FIG. 17). A recent CRISPR screen in U937 cells (monocytic, from pleural fluid of a patient with histiocytic lymphoma) confirmed CD18 and its extracellular domain as important for toxicity to these cells. However, forced expression of CD18 in other cells does not predictably impart HlyA sensitivity, indicating that toxin-receptor interactions are complex and likely cell type-specific. HlyA also exhibits toxicity to epithelial cells, though these have not been as extensively studied. HlyA can elicit pyroptosis and inflammatory cytokine release in vitro from renal and bladder epithelial cells. In cultured 5637 bladder epithelial cells, infection with HlyA-expressing UPEC at sublytic concentrations led to protease activity in the cytoplasm associated with degradation of paxillin and other proteins, and activation of NF-kB signaling; these effects were significantly lessened during infection with an hlyA mutant. A recent paper also showed that HlyA promotes escape of internalized UPEC from vesicles within 5637 cells. As epithelial cells do not express integrins (such as CD18) on their surfaces, it is surmised that HlyA must interact with these cells in ways not previously known. Recent work, summarized in Example 1 and below in the Example 2 Results section, identified a novel mechanism of action for HlyA against epithelial cells. Specifically, HlyA-mediated cytotoxicity required clathrin-mediated endocytosis (CME) and interaction with LDLR, and HlyA internalization led to lysosomal membrane permeabilization (LMP), mitochondrial dysfunction, and rapid caspase-independent cell death. In total, the published literature indicates that HlyA may have multiple binding partners and/or modes of action, likely depending on target cell type. In Example 2, in the context of the new paradigm for HlyA function via endocytosis, its interactions with two cell populations that are critically important in sepsis pathophysiology, myeloid cells and vascular endothelial cells, are interrogated.

The contribution of HlyA to pathology in E. coli sepsis is largely unexplored, though small studies indicate the hlyA gene is carried by up to 35% of human bloodstream E. coli isolates. One recent but limited study showed that compared with a nonpathogenic K-12 E. coli isolate, one carrying hlyCABD on a plasmid caused increased mortality after intravenous inoculation in mice. That same paper showed higher circulating proinflammatory cytokine levels as well as thrombocytopenia within a few hours after inoculation of the HlyA-expressing E. coli strain. HlyA has been investigated somewhat more in UTI pathogenesis, where ˜80% of UPEC isolates from patients with pyelonephritis encode HlyA, compared to only 40% of cystitis isolates. In preclinical models of cystitis, a UPEC mutant overexpressing HlyA accelerates shedding of bladder superficial cells, thereby resulting in reduced bacterial loads in the bladder. At sublytic concentrations, HlyA was shown to cause exfoliation and apoptosis of bladder superficial cells, perhaps by stimulating cleavage of host cytoskeletal proteins. However, genetic deletion of hlyA did not alter bladder UPEC loads at acute time points, nor the establishment of chronic cystitis. In a model using two-photon microscopy in live mice, microinjection of individual renal tubules with HlyA-expressing UPEC caused localized renal tubular cell damage and altered initial leukocyte recruitment. More recently, work in Example 1 detailed the contribution of HlyA to renal tissue injury during ascending pyelonephritis, even while kidney bacterial loads were unaffected by HlyA presence or absence. In Example 2, the novel mechanistic paradigm for cellular interaction with HlyA is brought to bear on the pathogenesis of sepsis and future development of anti-virulence interventions.

### LDLR and its Participation in Microbial Pathogenesis

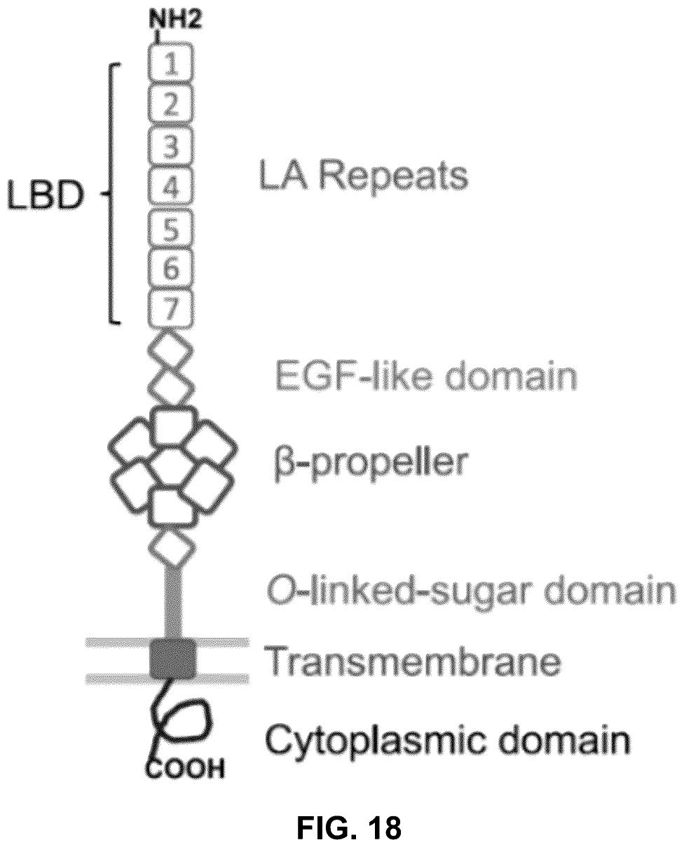

LDLR is broadly expressed across most cell types in humans, mice, and other mammals. It is the prototypic member of a family of cell-surface glycoprotein receptors (FIG. 18) that also includes very low-density lipoprotein receptor (VLDLR), LRP8 (aka ApoER2), LDLR-related protein 4 (LRP4, aka MEGF7), LRP1, LRP1b, and LRP2 (aka megalin). Beyond this core family, there are other related mammalian proteins that share domain structure with LDLR. Clathrin-mediated endocytosis and LDLR family members have been implicated in the pathogenic activity of several microbial toxins, though from distinct toxin families (not the RTX family). For example, activity of Clostridioides difficile exotoxin TcdA (a member of the large Clostridium toxin [LCT] family) is reduced in epithelial cells lacking LDLR, though direct TcdA-LDLR interaction was not observed, suggesting LDLR might have a facilitating role after initial TcdA binding to surface glycosaminoglycans. Several LDLR family members (LDLR, LRP1, megalin) act via their ligand-binding domains to promote internalization of Clostridium novyi alpha-toxin (Tcna). LRP1 also serves as a receptor for the uniquely structured vacuolating cytotoxin (VacA) of Helicobacter pylori, Pseudomonas aeruginosa exotoxin A (an ADP-ribosylating toxin), and the deaminase toxin of Pasteurella multocida. Anthrax toxin, an example of the A-B toxin class, gains cellular entry via the extended LDLR family member LRP6. Importantly, while these examples illustrate how LDLR family members participate in microbial toxin internalization, they belong to toxin classes—such as enzymatic or A-B toxins—that are known to require entry into the cytoplasm to exert their effects. In contrast, the findings with HlyA, detailed in Example 1 and the Results section below, represent the first demonstration of a suspected pore-forming toxin requiring internalization via LDLR-mediated, clathrin-dependent endocytosis in order to exert its cytotoxic activity. Of note, LDLR family members are also co-opted for cellular entry by certain viruses, including encephalitic alphaviruses, vesicular stomatitis virus, and hepatitis B virus.

### Pathogen and Host Drivers of Sepsis

Sepsis can be defined as life-threatening organ dysfunction associated with a dysregulated host response to infection. In humans, infections of the bloodstream, lung, abdomen, or other sites with Gram-negative (e.g., E. coli, Klebsiella, P. aeruginosa) or Gram-positive (e.g., Staphylococcus aureus, Streptococcus pyogenes, Enterococcus spp.) bacteria are frequent etiologies of sepsis. Physiologic manifestations of sepsis include fever, hypotension, poor tissue and organ perfusion, vascular permeability and edema, respiratory compromise, systemic inflammation, and disseminated intravascular coagulation (DIC). The entirety of decades of research into sepsis pathophysiology cannot be elaborated here (many extensive review articles have been published), but here the salient points relevant to Example 2 are summarized. Current understanding is that pathogen-associated molecular patterns (e.g., LPS, peptidoglycan and lipoteichoic acid of the bacterial cell wall) and bacterial toxins activate receptors (e.g., Toll-like receptors) on a variety of cell types to initiate antimicrobial and inflammatory signaling pathways, leading to cytokine secretion, immune cell recruitment and activation, and cellular injury. Among the many cell populations that are affected by and participate in sepsis pathophysiology are immune cells, particularly those of the myeloid lineage (monocytes, neutrophils, macrophages) and endothelial cells (ECs), which line the vasculature throughout the body's circulatory system and end-organ microvasculature. Endothelial cells express PAMP and cytokine receptors and are thereby activated by microbial products and circulating inflammatory cytokines. These signals cause ECs to express and secrete further pro-inflammatory cytokines, pro-coagulant factors, and pro-adhesion molecules. Cytokines and pro-adhesion molecules drive myeloid cell activation, adherence to capillary endothelium, and diapedesis into tissues, where these immune cells drive and amplify end-organ inflammation. Cell-junctional proteins in the endothelium are also cleaved, and there is degradation of the glycocalyx (a gel-like layer normally overlying the endothelial surface); these processes enable capillary leak and the diffuse edema often observed in patients with sepsis. Endothelial damage, coupled with marked EC expression of pro-coagulant factors (e.g., tissue factor) and anti-fibrinolytic factors (e.g., plasminogen activator inhibitor 1 [PAI-1]), leads to widespread exuberant blood coagulation in small vessels and capillaries (manifesting as DIC), causing ischemia to end organs and tissues. Of note, candidate therapeutics arising from the current molecular understanding of sepsis pathways have not yet been successfully translated to human use. The most familiar such agent is activated protein C (APC), which exerts anti-coagulant effects through inhibition of Factors Va and Villa as well as anti-inflammatory and endothelial cytoprotective effects. Though initial trials of APC were promising, a benefit to mortality was not confirmed in subsequent trials, and bleeding complications were frequent, leading ultimately to the drug's withdrawal. It is noted that these trials enrolled subjects with sepsis caused by a broad range of pathogens, ignoring the emerging concept that the host-pathogen interactions and cellular injuries in sepsis vary across causative species. It is now becoming clear that sepsis must be classified on the basis of specific pathogens and virulence factors (such as toxins) in order to enable development of targeted and effective adjunctive therapies.

Germane to this proposal, ˜30% of sepsis in humans arises from UTIs, the majority of which are caused by E. coli. Most preclinical modeling of Gram-negative sepsis has been done with injections of LPS. These studies have yielded significant insights but do not capture the entirety of the host-pathogen interaction, including the effects of specific virulence factors such as HlyA. The mouse model of UPEC sepsis, described herein elsewhere below, enables dissection of the importance of HlyA to sepsis morbidity and mortality, and provides novel and important insights into toxin-host cell interactions, whole-animal pathophysiology, and therapeutics development in E. coli sepsis. In Example 2, cellular and molecular drivers of E. coli sepsis are interrogated through the lens offered by recent discoveries in Example 1. Specifically, mechanisms of toxicity of HlyA to key cell populations participating in sepsis are defined, namely myeloid cells and ECs. How LDLR, CD18, and CME participate distinctly or cooperatively in HlyA activity against monocytes and neutrophils is dissected, and detailed studies of HlyA interaction with ECs and the consequences of this interaction on sepsis-relevant injury and activation of ECs are performed. Finally, how blocking HlyA-LDLR and/or HlyA-CD18 interactions can mitigate the myeloid and EC activation and damage that underlies sepsis is tested in vivo, using conditional knockout (KO) mice and candidate therapeutic agents based on LDLR, APOB (canonical LDLR ligand that is the protein component of LDL particles), or CD18.

### Innovation

The work described in Example 2 provides extensive conceptual innovation by defining the mechanisms by which HlyA exerts toxicity to sepsis-relevant cell types (monocytes, neutrophils, and ECs), enhancing the specific knowledge of E. coli virulence strategies and broadening understanding of bacterial toxin activity against mammalian cells. Recent discoveries implicating LDLR and CME in HlyA function are reconciled with prior work on CD18 by dissecting how these molecules work separately or in concert to enable HlyA toxicity to myeloid cells. At the same time, the molecular and cellular effects of HlyA on ECs have been relatively ignored—a surprising fact given the recognized importance of ECs to sepsis pathophysiology. Moreover, Example 2 addresses a critical clinical need—to understand sepsis pathophysiology according to the causative pathogen and its virulence arsenal, rather than lumping all sepsis patients into one bin—with the ultimate goal of developing pathogen-specific adjunctive therapies. Example 2 offers technical innovation through an updated sepsis model that utilizes whole E. coli bacteria (rather than the typically used LPS injection), enabling interrogation of specific virulence factors (in this case HlyA) and the identification of host cell types whose protection from HlyA will reverse sepsis morbidity and mortality. In addition, as described below, the studies utilize fresh HlyA-rich conditioned medium (CM) prepared daily for experiments and normalized for activity via daily rabbit erythrocyte assays. Though labor-intensive to prepare and standardize each day, this reagent is more consistent in its activity, and potentially more biologically relevant, than the acid-precipitated and reconstituted HlyA preparation used by other groups.

### Results

### Generation of Strains and Tools

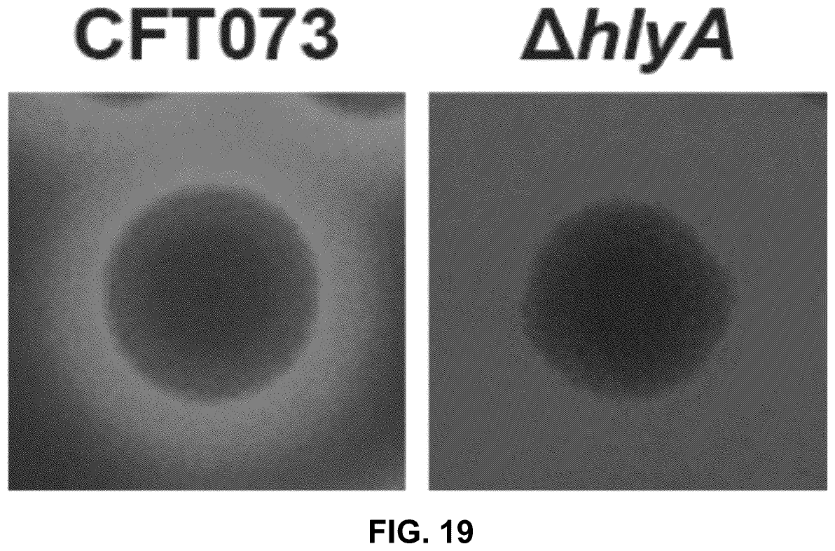

The genomically sequenced and oft-used model UPEC strain CFT073, like up to 80% of human UPEC isolates causing pyelonephritis, encodes the hlyCABD operon. CFT073 was isolated from the blood of a human patient with urosepsis and has been used extensively in mouse models and in vitro studies relevant to UTI. As done with the prototypic cystitis strain UT189, targeted deletion of hlyA in CFT073 was achieved using the lambda red recombination system and confirmed by direct sequencing; three independent CFT073 ΔhlyA clones were generated. Loss of hemolytic activity was confirmed on blood agar plates (FIG. 19); lack of hlyA transcript was verified by qPCR, and ΔhlyA mutants grew normally in laboratory media. Multiple strategies to cleanly complement hlyA in situ within the chromosomal operon were unsuccessful, reflecting complex transcriptional regulation of the operon. Of additional note, though an HlyA-overexpressing plasmid (pSF4000) is useful for certain in vitro studies, plasmid retention during in vivo mouse experiments cannot be relied upon for the requisite time frames (days).

As noted above, purification of HlyA for biochemical studies, structure determination, and/or antibody generation has been attempted by multiple groups with limited success. The Welch group published a method to generate HlyA-rich, sterile conditioned medium (HlyA-CM) from E. coli broth culture, in which >90% of the protein content is HlyA. This method was optimized to generate HlyA-CM from broth culture of UT189/pSF4000 or ΔhlyA and to normalize its hemolytic activity (i.e., its HlyA content) with a rabbit erythrocyte assay, as there is minor day-to-day variability among CM preparations (all CM are used immediately following preparation).

### Clathrin-Mediated Endocytosis and LDLR Mediate HlyA Toxicity to Renal Epithelial Cells.

See also Example 1.

### HlyA as a Virulence Factor in UPEC Sepsis.

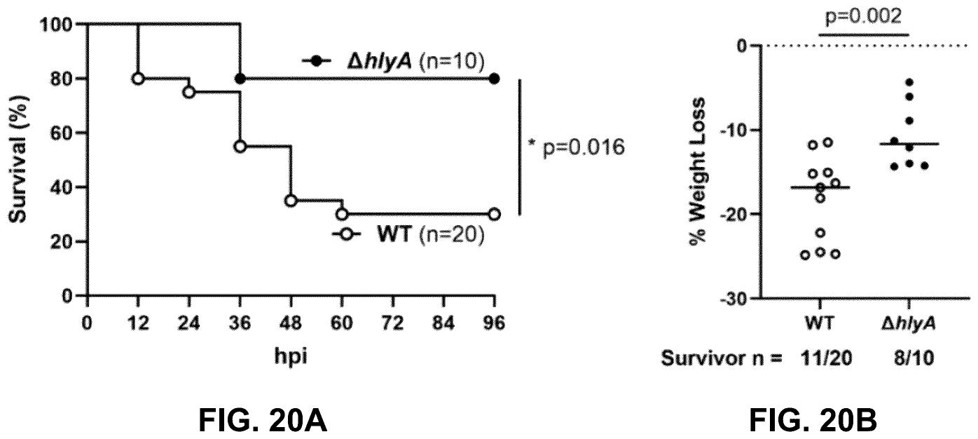

To begin investigating HlyA as a mediator of pathology in E. coli sepsis, a mouse model was developed. After inoculation of 8-week-old male C57BL/6 mice with 108 colony-forming units (CFU) of UPEC strain CFT073 via the retroorbital route, mortality was 70% within 96 h (FIG. 20A). Importantly, infection with CFT073 ΔhlyA yielded only 20% mortality (*p=0.016, FIG. 20A), significantly less weight loss among survivors (**p=0.002, FIG. 20B), and lower sepsis behavior scores. Further data from initial murine sepsis experiments are described herein below.

In total, the present Example has unveiled a novel mechanism of action for HlyA, one of the “oldest” but more enigmatic bacterial toxins. The findings in epithelial cells prompt fresh consideration of the functions of RTX toxins in general, and of HlyA specifically. Additional studies relevant to UTIs are separately pursued, but in the present Example, the new paradigm has been applied to an even more serious human disease—sepsis—and to new cell types that are directly relevant to sepsis pathophysiology. This work produces new and detailed insights into HlyA function, finally clarifying its proposed mechanism of action against myeloid cells (Aim 1). Moreover, there are few, decades-old studies of HlyA toxicity to ECs—perhaps the most critical cell population to understand in developing future specific therapies for sepsis—and the cellular and molecular basis of EC-HlyA interaction is defined (Aim 2). Finally, these results in the mouse sepsis model give high confidence of in vivo correlation of these host-pathogen interactions and their therapeutic targeting.

### Redefining the Mechanisms of HlyA Toxicity to Myeloid Cells.

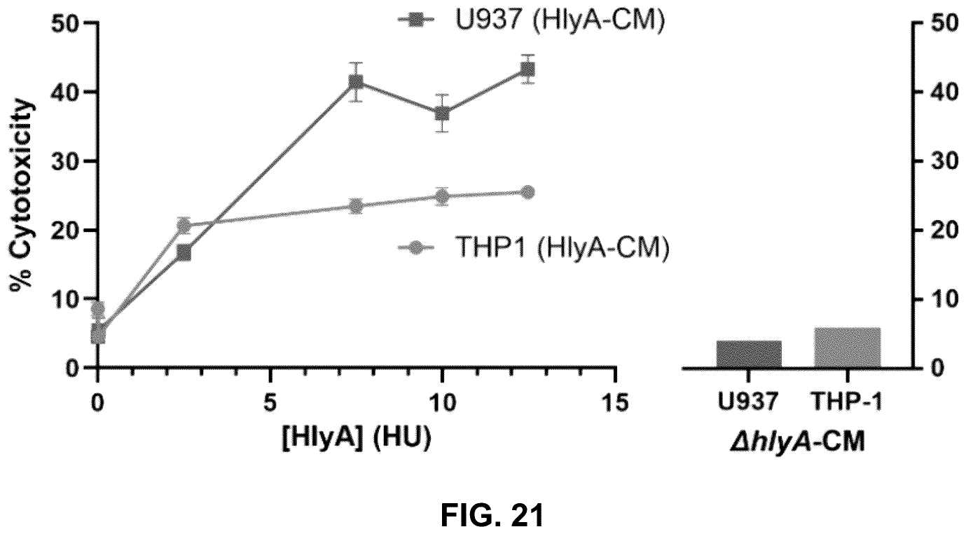

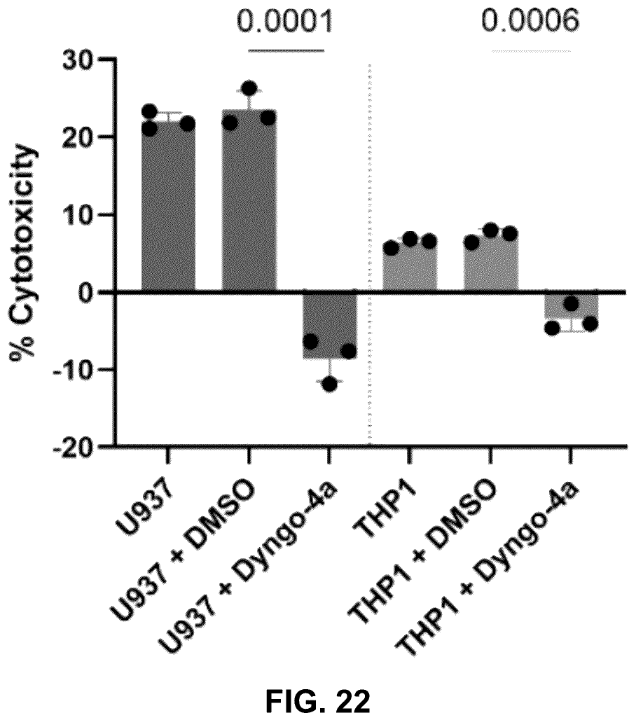

Published studies argue that the β2-integrin CD18 is a receptor for HlyA on macrophages, enabling HlyA to form pores in the plasma membrane. In the most recent study supporting this concept, a CRISPR mutant screen was performed in the U937 (monocytic) cell line, using an HlyA-enriched preparation made by precipitation and re-dissolution of proteins from CFT073 conditioned medium. The ITGB2 gene (encoding CD18) was the only statistically significant hit in that screen; notably, LDLR and genes related to CME were not hits, a curious result as U937 cells do express LDLR. Conversely, because epithelial cells do not express CD18, Itgb2 was logically not a hit in the screen of IMCD-3 cells exposed to HlyA-CM (FIG. 2). As described, there is data indicating that HlyA toxicity to U937 and another myeloid cell line, THP-1 (macrophage-like), is dose dependent (FIG. 21) and relies on endocytosis (FIG. 22). These data suggest that instead of (or in addition to) forming plasma membrane pores upon interaction with CD18, HlyA can exert toxicity via internalization, as CD18 and other integrins are reported to undergo dynamin-dependent endocytosis that can be clathrin dependent or independent. To disentangle and clarify the collective literature on HlyA and myeloid cells, (i) whether endocytosis (clathrin-mediated or otherwise) of either or both of the putative receptor CD18 and LDLR are at play in these cell types, and (ii) the potential for multiple or parallel mechanisms of HlyA-mediated cell injury/death in myeloid cells, are interrogated. Taking into account all the available data, endocytosis of HlyA via CD18 and/or LDLR mediates its toxicity to myeloid cells. Whether HlyA killing of myeloid cells arises from HlyA internalization with ensuing LMP and mitochondrial dysfunction (as found in epithelial cells), rather than plasma membrane poration, can be further specified. These results provide the field with much-needed clarity regarding HlyA mechanism(s) of action on myeloid cells.

### Experimental Design

### Establish the Susceptibility of THP-1 and U937 Cells to HlyA.

HlyA-CM from CFT073 are prepared and dose-response curves of cytotoxicity for THP-1 (differentiated with phorbol myristate acetate [PMA]) and U937 cells (with or without PMA, which promotes adhesion) are generated. Notably, THP-1 cells adopt a more M1-like macrophage phenotype on activation, while U937 cells exhibit a more M2-like phenotype. As done previously, HlyA-CM are generated daily and the hemolytic activity of these preparations are normalized using a rabbit erythrocyte assay. Preliminary data (FIG. 21) show dose-dependent killing of these myeloid lines by HlyA-CM, with minimal cell death in the presence of CM prepared identically from ΔhlyA (denoted ΔhlyA-CM), which serves as a control condition for the experiments below. Then, dose-response curves for both cell lines can be established.

### Establish Mechanisms and Phenotypic Features of HlyA-Mediated Cell Death in U937 Cells.

A requirement for endocytosis can be tested both pharmacologically and genetically. U937 cells are treated with the dynamin inhibitor Dyngo-4a (in serum-free medium) prior to HlyA-CM exposure and measure protection from HlyA-mediated cytotoxicity (as seen in IMCD-3 cells; FIGS. 2D and E). On a technical note, serum inactivates Dyngo-4a, so medium is changed to serum-free before Dyngo-4a is added. An initial experiment indicates complete protection of U937 cells by Dyngo-4a (FIG. 22, blue bars). Expression of key CME genes (CLTC and AP2M1) in U937 cells are knocked down by siRNA (oligos generated in the Origene Trilencer-27 system) and protection from HlyA-mediated killing is measured (as done in IMCD-3 cells; FIGS. 2F and G). Of note, published works indicate that standard methods for siRNA transfection (e.g., electroporation or typical lipid transfection reagents) are inefficient or overly toxic in U937 cells. However, the multiple successful methods have been reported with these cells can be adopted: differentiation of U937 cells with PMA, use of DharmaFECT transfection reagent, siRNA packaging in the HVJ Envelope vector, or use of U937 Cell Avalanche transfection reagent (EZ Biosystems) made for this cell line.

It is particularly important to discern the contribution of CD18, as it is implicated in HlyA toxicity to this cell line in particular; of note, α-integrin components (FIG. 17) have no effect on HlyA function. To determine whether CD18 is needed for HlyA toxicity to U937 cells, ITGB2 expression is knocked down by siRNA prior to treatment with HlyA-CM. Again, CRISPR disruption of ITGB2 is an alternative strategy, as was done by Welch. Pre-treatment of U937 cells with anti-CD18 monoclonal antibodies (mAbs) is also tested. Several mAbs recognizing CD18 have been shown to block HlyA cytotoxicity to HL-60 neutrophil-like cells and are commercially available (e.g., R3.3, KIM127). To determine if HlyA interaction with LDLR is required for U937 cell killing, LDLR expression is knocked down by siRNA and test HlyA susceptibility. As an alternative to siRNA, CRISPR disruption of LDLR can be generated in these cells, as done in IMCD-3 cells (FIGS. 4C and D). Whether addition of an APOB-derived peptide (as in FIG. 5C) or soluble LDLR-Fc (as in FIGS. 5D and E) inhibits HlyA toxicity to U937 cells is also tested. If needed based on the above results, both LDLR and CD18 (via combination of multiple siRNAs, CRISPR modifications, or mAbs) can be dampened or blocked to discern whether there is redundancy between CD18 and LDLR as receptors for endocytic internalization of HlyA.

If LDLR- or CD18-mediated HlyA internalization is active or predominant in these cells, as seen in renal epithelial cells, evidence of LMP and mitochondrial injury is expected. Therefore, LMP (by LysoTracker Red and the galectin puncta assay); cytoplasmic acidification (by pHrodo Green and by cathepsin-D immunoblot on the cytoplasmic fraction from cell lysates); and mitochondrial injury by MitoTracker Green (measures mitochondrial mass), MitoTracker Red (reflects mitochondrial mass and membrane potential), and TMRM (measures mitochondrial membrane potential) are all ascertained. Mitochondrial injury is also assayed by production of reactive oxygen species (mROS), using MitoSOX dye. To corroborate the MitoTracker and TMRM data, the impact of sublytic HlyA concentrations on mitochondrial respiration is quantified, measuring the oxygen consumption rate (OCR) using the Agilent Seahorse XFe96 system. This system enables real-time OCR measurements with precisely timed addition of HlyA-CM (at varying concentrations), dyes, or inhibitors (e.g., Dyngo-4a) into specific wells as needed, and DAPI staining with analysis on the adjacent Agilent Cytation 5 cell imager controls for cell viability.

If CME-mediated HlyA internalization via LDLR or CD18 is active or predominant in these cells, it is further expected that cell death (likely autolytic/necrotic, as can arise from LMP) will be caspase independent, as seen in epithelial cells. If instead the historical model of plasma membrane poration via HlyA-CD18 interaction is active (whether at all or only some HlyA concentrations), caspase activation is expected (which can be measured by immunoblot, for example, for pro-caspase-1 and cleaved caspase-1), inflammasome activation (evidenced by secretion of IL-1p and IL-18), and microscopic evidence of pyroptotic cell death. Indeed, HlyA-mediated death of THP-1 cells has been reported to activate caspase-1. The listed caspase and inflammasome activation markers in the presence of varying HlyA exposures (concentration and time) and with the LDLR-, CME-, and CD18-targeted perturbations outlined above are measured, and broad or narrow caspase inhibitors are introduced, in order to clarify the mode(s) of HlyA-induced myeloid cell death. Of note, one study in human monocyte-derived macrophages proposed multiple modes of cell death depending on HlyA concentration.

Finally, internalization is demonstrated and cellular localization of HlyA (cell membrane vs associated with various intracellular organelles) using immunoblots of cell fractions with anti-HlyA antibody H10 is specified. Ongoing attempts to localize the toxin by immunofluorescence (IF) microscopy can also be performed, which has not been accomplished in the literature; HlyA are currently cloned with various tags, with the goal of amplifying HlyA signal while not interfering with toxin secretion and function.

Establish mechanisms and phenotypic features of HlyA-mediated cell death in THP-1 cells Given that THP-1 cells have been used to interrogate HlyA function in multiple papers, and in the context of the new findings herein implicating endocytosis in HlyA toxicity to these cells (FIG. 22, green bars), THP-1 cells are tested with the suite of experiments and analyses outlined above. Specifically, pharmacologic inhibition of CME, siRNA knockdown of AP2M1, CLTC, LDLR, and ITGB2, CRISPR gene disruptions if needed, and cytotoxicity inhibition by APOB, LDLR-Fc, and/or anti-CD18 mAb are pursued. In parallel with the U937 studies, investigations of LMP, mitochondrial dysfunction, and cell death pathways are also undertaken as outlined above.

### Interrogate Toxicity of HlyA-CM to Human Neutrophils

Beyond the myeloid cell lines above, HlyA causes cell death in neutrophil-like differentiated HL-60 cells. However, studies of interactions between HlyA and actual human neutrophils (polymorphonuclear leukocytes [PMN]) are few. With the novel insights into HlyA biology as described herein, and history of working effectively with human PMNs and their functions (including degranulation, phagocytosis, transmigration, and chemotaxis), this disclosure illuminates the HlyA-PMN interaction with translatability to human disease. These experiments require close coordination of HlyA-CM preparation and the isolation of human PMNs, as these PMNs have a useful lifespan measured in hours after purification. On the day of each experiment involving PMNs, while HlyA-CM is being prepared in parallel by other staff, blood is drawn from donors (see Human Subjects section) and PMN isolated using a standard methodology, based on 3% dextran sedimentation, Ficoll gradient centrifugation, and hypotonic erythrocyte lysis. The yield from a single blood draw of 20 mL is ˜2×10⁷ PMNs, plenty for assays. Susceptibility curves are generated with a range of HlyA-CM doses, against PMNs isolated on different days from several healthy donors (male and female), using ΔhlyA-CM as control.

Though well known for their phagocytic activity, PMNs also undertake a significant degree of endocytosis to support basic cellular functions as well as certain antimicrobial activities. Specifically, CME is necessary for exocytosis of granules and for completion of the respiratory burst (activity of NADPH oxidase). Accordingly, a requirement for CME in HlyA cytotoxicity to PMNs is tested using Dyngo-4a, but it is anticipated that Dyngo-4a would act to diminish granule exocytosis and ROS production.

To test a requirement for CD18 in HlyA toxicity to human PMNs, anti-CD18 mAb is utilized, as siRNA transfection in fresh PMNs is fraught with challenges (poor transfection efficiency, short PMN half-life, and unintended PMN activation). Bone marrow-derived neutrophils (BMDNs) are also obtained from mice lacking CD18 in the germline (C57BL/6 Itgb2−/−, Jax #037246) or on PMNs specifically, and their susceptibility to HlyA compared with neutrophils isolated from WT C57BL/6 mice is tested. Because Itgb2−/− mice phenotypically mimic human leukocyte adhesion deficiency type I, circulating neutrophil counts will be elevated; therefore, sufficient PMN from peripheral blood of Itgb2−/− mice can be obtained to perform HlyA cytotoxicity assays, recognizing that some of these cells' functions, such as adhesion and respiratory burst, will be impaired. PMNs also express LDLR and other family members, and LDLR involvement can be tested using LDLR-Fc or APOB peptide as described above, or with BMDNs from Ldlr−/− mice.

Beyond cell death, how exposure to sublytic HlyA concentrations affects the antibacterial functions of PMNs, and how CD18 and/or LDLR mediate such effects, can be defined. In one of the 1990s papers, whole E. coli expressing HlyA were more resistant (compared with those lacking HlyA) to intracellular killing by human PMNs. Here, in human PMNs, degranulation (by flow cytometry for exocytosis markers) and phagocytosis of GFP-expressing HlyA-negative E. coli (by standard fluorescence-based assays) in the presence or absence of low concentrations of HlyA, and with or without anti-CD18 mAb or LDLR-Fc, are quantified.

Given the preliminary result in FIG. 22, the most likely outcome is that CD18 enables HlyA toxicity to myeloid cells via endocytosis, leading to lysosomal and mitochondrial injury and resultant cell death. This result represents a major shift from the established model for how CD18 participates in HlyA function. If Dyngo-4a blocks HlyA toxicity (FIG. 22) but CME-specific siRNA knockdowns (CLTC, AP2M1) do not, this implicates an alternative internalization pathway for HlyA such as caveolar endocytosis, as suspected in one study of Aggregatibacter LtxA. If the balance of potential plasma-membrane and endocytic mechanisms is related to HlyA concentration, and/or relative expression of CD18 and LDLR, this can be revealed by the described assays, which use a range of HlyA exposures (concentration and time). If endocytosis is required but blockade of either LDLR or CD18 does not mitigate toxicity, this indicates either a cooperative interaction requiring both CD18 and LDLR, or less likely a yet-unidentified third receptor for HlyA. If a cooperative interaction is suspected, further studies can seek to confirm this biochemically and to specify participating domains of LDLR (FIG. 18) with constructs that are in hand. Ultimately, this portion of Example 2 resolves prior work suggesting CD18 enables HlyA poration of myeloid cell membranes with the new paradigm and preliminary data that implicate endocytosis.

In some aspects, a commercially available anti-LDLR Ab that blocks HlyA toxicity can be identified. In separate studies, which domains of LDLR participate in HlyA binding are systematically tested; results of that work can inform generation of an antibody that blocks HlyA-LDLR interaction. Instead of LDH assays for cytotoxicity, MTT or resazurin assays can be utilized. It is possible that on one or more of the myeloid cell types, other LDLR family member(s) can also mediate HlyA toxicity; this can be tested separately in the IMCD-3 model, and constructs to express various LDLR variants and family members are included in the present disclosure. To identify additional host mediators of HlyA toxicity to myeloid cell lines, CRISPR mutant screens can be performed as done in IMCD-3 cells (FIGS. 2A, 2B, and 15); to further discern cellular effects of HlyA, RNAseq can be pursued on cells treated with sublytic HlyA concentrations. Instead of fresh human PMNs, differentiated HL-60 cells, in which case siRNA and CRISPR approaches are more accessible, can be utilized. HlyA toxicity to myeloid cells is linked to CD18 in mouse cells as well as human, so minor sequence differences between mouse and human CD18 are not expected to complicate the murine PMN experiment; however, if needed, hCD18 knock-in mice can be utilized for this experiment (C57BL/6-Itgb2^{tm1.1(ITGB2)Kley}/J, Jax #037426).

### Define the Mechanisms of HlyA Toxicity to and Activation of Endothelial Cells.

Current understanding of HlyA interaction with and effects on ECs is based largely on a handful of papers from the 1990s. HlyA was shown to promote permeability of cultured EC monolayers and edema in isolated perfused rabbit lungs. In the former study, cell retraction and appearance of “intercellular gaps” were noted by microscopy. HlyA elicits nitric oxide (NO) release by ECs, and promotes the adhesion of human PMN to cultured human umbilical vein endothelial cells (HUVECs). In that paper, ECs exposed to HlyA did not upregulate the adhesion molecules ICAM-1 or VCAM-1, though expression of CD11 b/CD18 (aka Mac-1) was increased. HlyA was also shown to activate EC production of platelet-activating factor (PAF, a phospholipid mediator of platelet aggregation that also promotes inflammation and vascular permeability). In total, these papers relate some phenotypic effects of HlyA on ECs but do not specify the surface receptors, signaling mechanisms, or cellular mediators of these effects, nor how they impact sepsis. In this section of Example 2, HlyA intoxication of ECs via LDLR-mediated CME leading to EC death and barrier dysfunction (permeability) is tested, as well as activation of pro-inflammatory and pro-adhesion mediators that promote sepsis pathology. In vitro studies are pursued with two types of cultured endothelial cells: (i) EA.hy926 cells (ATCC #CRL-2922)—an immortalized vascular endothelial cell, created by fusion of a HUVEC and A549 cell, routinely used in cardiovascular research; and (ii) primary human lung microvascular endothelial cells (HMVEC-L; Lonza #CC-2527), which is diploid and may better reflect the microvascular location of endothelial injury during sepsis.

### Establish Mechanisms of EC Death and Activation in EAhy.926 Cells.

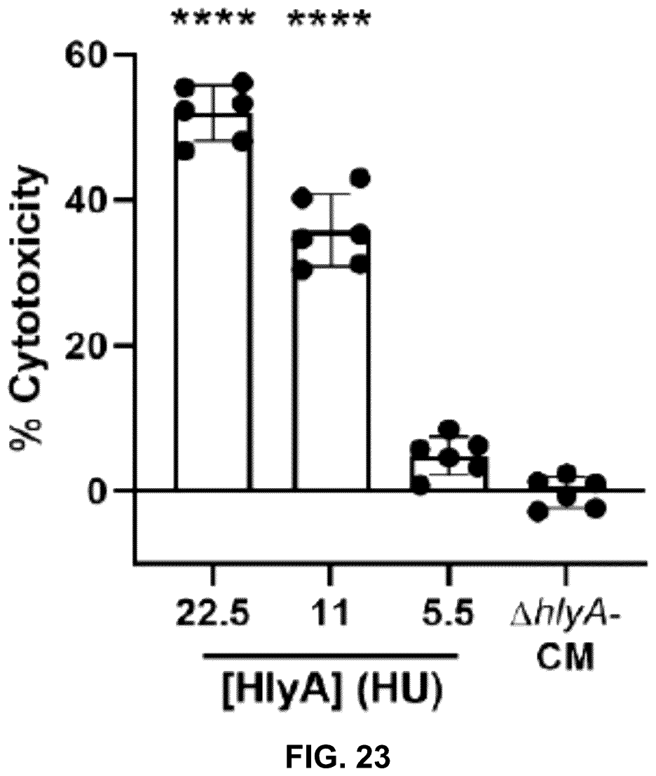

To establish the mechanism(s) of HlyA-induced EC death and identify host mediators, experiments analogous to those outlined for myeloid cells in the previous section are first undertaken. Initial experiments demonstrate dose-dependent death of EAhy.926 cells (grown in Dulbecco's modified Eagle medium) on exposure to HlyA-CM (FIG. 23). To test the involvement of CME and LDLR in HlyA-mediated killing, pharmacologic inhibition of endocytosis is repeated and siRNA knockdown of AP2M1, CLTC, and LDLR, CRISPR gene disruptions, and inhibition by APOB and/or LDLR-Fc, can be pursued as in the previous section. Of note, siRNA transfection of EAhy.926 cells with typical reagents is reported in multiple papers. Regarding CD18, reports regarding its expression by EAhy.926 cells are mixed; at least one paper indicates that CD18 is expressed (as the CD11c/CD18 heterodimer) by these cells, but others failed to confirm this. Thus, CD18 expression is quantified by EA.hy926 cells, using ITGB2 qPCR, immunoblot of cell lysates, and IF microscopy with anti-CD18 Abs. If CD18 expression is confirmed, its participation in HlyA-induced death of ECs can be interrogated by introducing ITGB2 siRNA, CRISPR modification, and/or CD18 mAb, as in the previous section. LMP, mitochondrial dysfunction, and cell death pathways are defined as outlined above. For microscopic and IF studies, ECs are grown on sterile chamber slides (Ibidi) that facilitate additions of toxin, other reagents, and stains to distinct wells, followed by direct microscopy (obviating the need for coverslip transfers to standard slides).

Activation of ECs elicits expression and secretion of mediators with pro-inflammatory, pro-coagulant, vasoactive, and pro-adhesive functions. As noted above, HlyA was shown to elicit EC secretion of the vasodilator NO, and to promote adhesiveness of ECs to nearby PMNs. A series of experiments are undertaken to study these effects and their mediators, while abrogating CME (with Dyngo-4a), LDLR (LDLR siRNA or CRISPR disruption, LDLR-Fc, or APOB) and/or CD18 (with ITGB2 siRNA, CRISPR disruption, and/or CD18 mAb), as listed below:
Pro-inflammatory: EC supernatant pro-inflammatory cytokines IL-6, IL-8, and MCP-1 are measured by commercially available ELISA or multiplex bead assay (Bio-Plex).
Pro-coagulant: EC supernatant tissue factor (TF; initiator of the extrinsic pathway of coagulation) and von Willebrand factor (vWF; promotes platelet adhesion and aggregation) are measured by ELISA. As needed, other pro-coagulant molecules secreted by activated ECs (e.g., PAF, thromboxane A2) can be measured in similar assays.
Pro-adhesion: Adherence of human PMNs to cultured ECs are assayed with mitigation of CME, LDLR, and/or CD18 as described above. To identify HlyA-elicited mediator(s) on ECs that enhance PMN adhesion, flow cytometry is used to quantify expression of ICAM-1 and VCAM-1 (comparing this to the published result) as well as E-selectin (a well-recognized neutrophil adhesion target) and P-selectin (which interacts more with platelets).

### Establish Mechanisms of EC Activation and Death in HMVEC-L Cells.

The suite of experiments and analyses outlined in the above section (Establish mechanisms of EC death and activation in EAhy.926 cells) are replicated, using HMVEC-L cells grown in phenol red-free VascuLife endothelial medium (Lifeline Cell Technology). These cells exhibit diminished lifespan (passage capacity) compared to the EAhy.926 cells but are more translationally relevant to the microvasculature, where the sepsis pathology related to ECs (end-organ ischemia and DIC) is taking place physiologically. As with the EAhy.926 cells, expression of LDLR and CD18 on HMVEC-L cells is determined. Cytotoxicity of HlyA-CM is tested in the context of pharmacologic inhibition of CME with Dyngo-4a, siRNA knockdown of AP2M1, CLTC, LDLR, and ITGB2, and cytotoxicity inhibition by APOB, LDLR-Fc, and/or anti-CD18 mAb. Of note, siRNA transfection of HMVEC-Ls with typical reagents has been reported previously. As outlined for EAhy.926 cells in the above section (Establish mechanisms of EC death and activation in EAhy.926 cells), investigations of LMP, mitochondrial dysfunction, and cell death pathways, and activation in HMVEC-Ls are also undertaken.

### Identify Mediators of HlyA-EC Interaction Leading to Vascular Permeability

As referenced above, EC injury leads to vascular permeability, which underlies the diffuse edema often observed in sepsis. In the 1990s work, an HlyA preparation applied to cultured ECs caused morphological changes (described as “intercellular gaps”) suggesting loss of cell-cell adhesion, and perfusion of isolated rabbit lungs with HlyA-containing medium caused rapid edema (reflecting vascular leak). Here, to interrogate the HlyA-EC interactions that underlie these changes, standard endothelial permeability assays using EC monolayers (EAhy.926 and HMVEC-L cells) are utilized. ECs are grown on collagen-coated Transwell inserts (0.4 μM pore) over 48-72 h, and confluence/impermeability is confirmed. Then, FITC-dextran is added to the upper chamber along with HlyA-CM or ΔhlyA-CM (control), and permeability is measured by FITC-dextran passage across the monolayer, by fluorescence intensity of medium sampled from the lower chamber at time intervals from 0 to 90 min. To corroborate the FITC-dextran results, electrical impedance is measured across EC monolayers upon HlyA exposure (i.e., TEER assays). Once an HlyA dose-response curve is established, and guided by results described above, perturbations outlined in earlier experiments to inhibit CME (Dyngo-4a) or HlyA interaction with LDLR (LDLR siRNA, LDLR-Fc, or APOB peptide) and/or CD18 (with ITGB2 siRNA, CRISPR disruption, and/or CD18 mAb) are introduced. Given the observation suggesting cell-cell junction damage with HlyA exposure, key cell-junctional proteins (e.g., VE-cadherin) are studied by IF microscopy and if loss of such a protein is identified, it is next tested whether HlyA exposure activates metalloproteinases (e.g., ADAM10) that cleave these proteins. Vascular permeability was also studied in a whole-mouse model described below.

Based on preliminary data (FIG. 23), it is anticipated that HlyA is internalized into ECs via CME, most likely using LDLR as a receptor. Accordingly, it is expected to observe EC cell death related to LMP and mitochondrial injury, and it is predicted that EC activation (including pro-inflammatory, pro-coagulant, and pro-adhesion activities) is inhibited by agents targeting LDLR or CME. As noted, expression of CD18 by these cells is possible (and is determined by the assays described herein); if present, CD18 might also function as a receptor (alone or in cooperation with LDLR). It is expected that EC activation elicits production of TF, vWF, and adhesion molecules (perhaps E-selectin, if ICAM-1 and VCAM-1 are not upregulated). It is expected that if ECs can be protected from HlyA toxicity (e.g., by Dyngo-4a or LDLR-targeting agents), the impermeability of EC monolayers can withstand HlyA-CM treatment.

As noted in above sections, as a backup or parallel approach to siRNA, CRISPR can be used to disrupt LDLR, ITGB2, or other target genes in EAhy.926 cells. It is possible that on one or both EC lines, another LDLR family member can mediate HlyA toxicity; again, constructs to express LDLR variants and family members can be used. To identify additional host mediators of HlyA toxicity to ECs, if necessary, CRISPR mutant screens can be performed (as in FIGS. 2A, 2B, and 15). To identify additional candidate pro-adhesive and pro-coagulant molecules expressed by activated ECs after HlyA exposure, bulk RNAseq can be performed on these cells, leveraging the Genome Technology Access Center (GTAC) at WashU. Differentially regulated genes of interest can be confirmed by qPCR, immunoblot, and IF microscopy, and expression (by siRNA or CRISPR disruption) or block activity (with Abs or pharmacologic inhibitors as available) of such candidates can be downregulated to prove their involvement. Instead of FITC-dextran, BSA-bound Evans blue dye can be used as an alternative permeability agent. Studying endothelial permeability in isolated mounts of murine vessels treated luminally with HlyA-CM can be considered; it is not proposed as a primary approach, as vessels required for this technique are necessarily large (aortic, mesenteric) and do not represent the microvascular environment where EC activation is most critical for sepsis pathophysiology. The Ibidi endothelial flow system can be used to dynamically flow HlyA-containing media over cultured ECs. This system can enable consideration of shear stress in EC injury from HlyA exposure.

Target Mediators of HlyA Action on Key Cell Populations to Mitigate E. coli Sepsis In Vivo.

The next step in translation is to demonstrate the in vivo correlates of the cellular and molecular findings in the mouse model of E. coli sepsis. It was found that there was a significant mortality difference in mice infected via retroorbital injection with E. coli CFT073 versus its isogenic ΔhlyA mutant (FIG. 20). Experiments in this section aim to interrogate how HlyA modulates myeloid cell and EC function during sepsis in the context of the whole animal. In addition, proof-of-concept for therapeutic targeting of relevant HlyA-host cell interactions to mitigate sepsis pathology (e.g., vascular permeability and DIC), morbidity (end-organ damage), and mortality is developed.

### Measure Dependency of Sepsis Outcomes and Vascular Permeability on HlyA Production

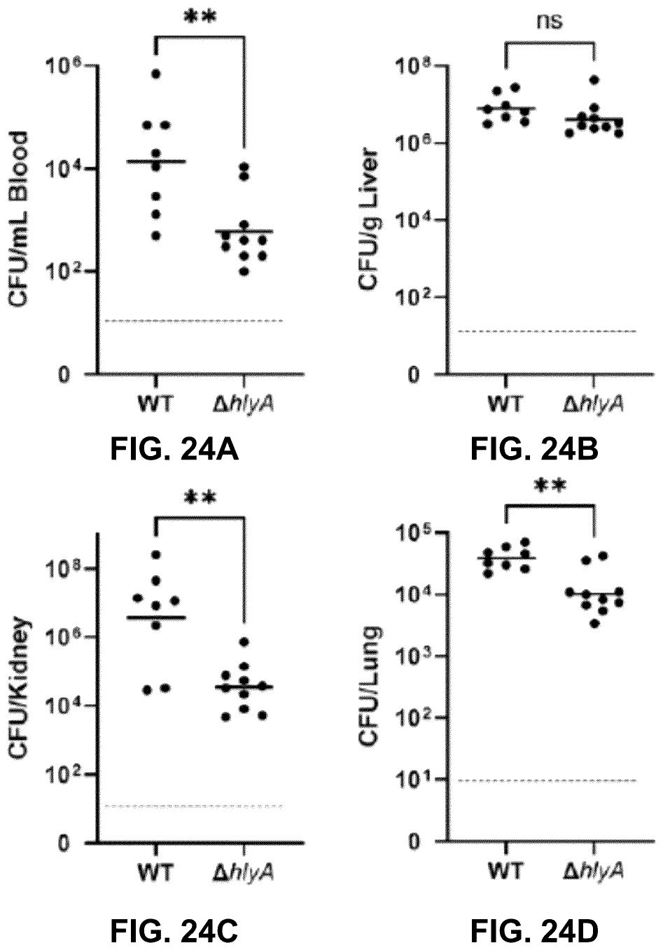

The end-organ effects of CFT073 sepsis, and their dependence on HlyA, are first established in the newly developed model. After retroorbital inoculation with 108 CFU CFT073, CFT073 ΔhlyA, or PBS (mock infection), twice-daily weights and sepsis behavior scoring are performed. Bacterial loads in blood and organs (liver, spleen, kidney, and lung) are measured 24 and 48 hpi (preliminary 24-hpi data shown in FIG. 24). Several organs (primarily lung and kidney; liver can also be used) are fixed, sectioned, and histologically examined to ascertain end-organ injury, vascular compromise, and evidence of DIC (to correlate with the EC phenotypes above). Intravascular clotting can be observed with standard H&E staining, but IF techniques can also be used to specify this finding. In separate mice, Evans blue dye administered i.v. 30 min before euthanasia is used to quantify vascular permeability, as has been done with other perturbations including LPS-mediated sepsis. After euthanasia, mice are perfused through the right atrium with acidic saline, then organs are harvested and bisected, and dye is extracted for 48-72 h with formamide before reading OD620 nm and calculating relative extravasation across infection conditions.

Test Mitigation of Sepsis with Agents Targeting HlyA Interactions with LDLR and/or CD18.

With prioritization guided by the results in earlier sections, candidate therapeutics (e.g., LDLR-Fc, anti-CD18 mAb, or APOB peptides) are introduced to reduce mortality and morbidity associated with CFT073 sepsis. Selected agent(s) may require dose-finding and verification of circulating half-life. Mice are pre-treated with selected agent(s) prior to CFT073 inoculation, twice-daily weights and sepsis behavior scoring are performed, and mortality and end-organ CFU and pathology as described above are ascertained. For an agent showing benefit in CFT073 sepsis, its specificity in infections with CFT073 ΔhlyA is confirmed, anticipating no benefit in this scenario.

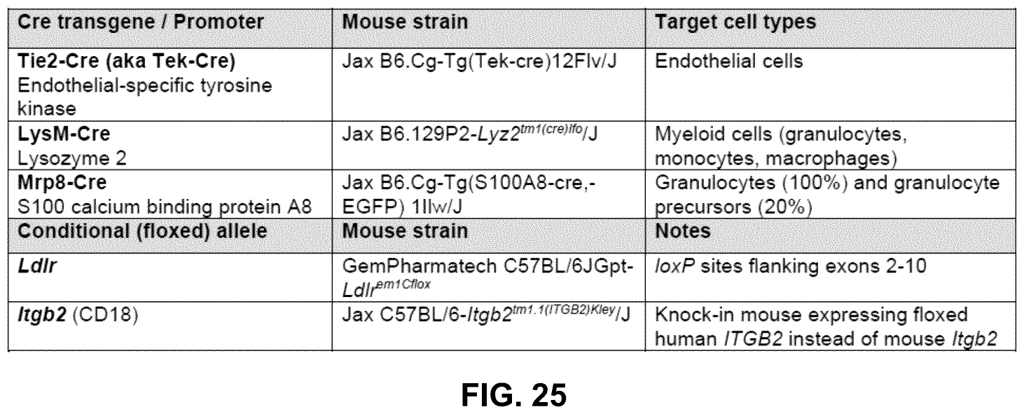

In parallel, a genetic approach is taken to specifying the contributions of LDLR and CD18 on myeloid and endothelial cells to the pathophysiology of E. coli sepsis. For these studies, germline Ldlr−/− mice (which model human familial hypercholesterolemia) are not used, as they are inherently highly susceptible to bacterial infections, including sepsis via cecal ligation and puncture. Similarly, studying sepsis in germline Itgb2−/− mice (which model human leukocyte adhesion deficiency) is not proposed as they develop spontaneous skin infections and exhibit not only defective neutrophil margination but also T cell dysfunction. Instead, mice with conditional deletion of genes of interest (Ldlr and Itgb2) are utilized in myeloid and/or vascular endothelial cells. C57BL/6 mice expressing Cre recombinase under selected promoters are crossed with mice carrying floxed Ldlr or Itgb2 (FIG. 25). All mice are commercially available. After retroorbital inoculation with 108 CFU CFT073, CFT073 ΔhlyA, or PBS (mock), weights, sepsis scores, mortality, and organ CFU and pathology are recorded as above.

It is anticipated that LDLR- and/or CD18-directed therapies lessen mortality in septic wild-type mice, depending on which of these HlyA receptors predominate in sepsis pathophysiology. If LDLR and CD18 both contribute (on different cell types), inhibition of one of these targets may not have optimal effect on mortality in wild-type mice. It is expected that the conditional KO experiments will illuminate which HlyA mediator, on which cell type, contributes most substantially to sepsis mortality and vascular/end-organ pathology in this model. These results can illuminate how a pathogen- or toxin-specific adjunctive agent might be translated to better therapeutic approaches to patients with sepsis in the future.

Deletion of CD18 on myeloid cell populations affects their ability to exit the vasculature to local sites of infection; organ titers in such mice will be interpreted with this in mind. If any of the above conditional KO mice prove to be nonviable (more likely with endothelial than myeloid deletions), inducible Cre constructs (e.g., VE-Cad-CreERT2 mice) can be used. As noted above, separate yet concurrent investigation includes which domain(s) of LDLR interact with HlyA. If such an LDLR truncate is identified, it is produced in larger quantities for in vivo testing. When male or androgen-exposed female C3H/HeN mice are given ascending pyelonephritis with CFT073, a minority of the mice develop bacteremia. In some embodiments, to further model mitigation of urosepsis, selected therapeutic agent(s) can be tested in this alternative mouse model. This “urosepsis” model is not primarily chosen in every embodiment due to the much larger numbers of mice required to test interventions and given the low sepsis rate after urinary inoculation. If desired to mimic clinical application, candidate interventions are tested as adjuncts with antibiotic therapy.

What is claimed is:

1. A composition comprising a soluble low-density lipoprotein receptor (LDLR)-Fc fusion protein, wherein the soluble LDLR-Fc fusion protein comprises:
an Fc domain; and
at least one LDLR type A domain.

2. The composition of claim 1, wherein the at least one LDLR type A domain comprises at least one of a ligand binding domain (LBD), LA1, LA2, LA3, LA4, LA5, LA6, LA7, and combinations thereof.

3. The composition of claim 1, wherein the at least one LDLR type A domain comprises at least one tandem domain selected from LA1-2, LA2-3, LA3-4, LA4-5, LA5-6, LA6-7, and combinations thereof.

4. A method of treating urinary tract infection (UTI) in a subject in need thereof, the method comprising administering to the subject a composition comprising an alpha-hemolysin (HlyA) inhibiting agent.

5. The method of claim 4, wherein the HlyA inhibiting agent comprises a soluble low-density lipoprotein receptor (LDLR)-Fc fusion protein comprising:
an Fc domain; and
at least one LDLR type A domain.

6. The method of claim 5, wherein the at least one LDLR type A domain comprises at least one of a ligand binding domain (LBD), LA1, LA2, LA3, LA4, LA5, LA6, LA7, and combinations thereof.

7. The method of claim 5, wherein the at least one LDLR type A domain comprises at least one tandem domain selected from LA1-2, LA2-3, LA3-4, LA4-5, LA5-6, LA6-7, and combinations thereof.

8. The method of claim 4, wherein the HlyA inhibiting agent comprises a clathrin-mediated endocytosis (CME) inhibitor.

9. The method of claim 4, wherein the HlyA inhibiting agent comprises an anti-LDLR antibody.

10. The method of claim 4, wherein administering the HlyA inhibiting agent protects against cell damage.

11. The method of claim 10, wherein the UTI is cystitis.

12. The method of claim 11, wherein the cell damage comprises at least one of bladder cell damage and bladder tissue damage.

13. The method of claim 10, wherein the UTI is pyelonephritis.

14. The method of claim 13, wherein the cell damage comprises at least one of renal cell damage and renal tissue damage.

15. A method of treating sepsis in a subject in need thereof, the method comprising:
administering to the subject a composition comprising an alpha-hemolysin (HlyA) inhibiting agent.

16. The method of claim 15, wherein the HlyA inhibiting agent comprises a soluble low-density lipoprotein receptor (LDLR)-Fc fusion protein comprising:
an Fc domain; and
at least one LDLR type A domain.

17. The method of claim 16, wherein the at least one LDLR type A domain comprises at least one of a ligand binding domain (LBD), LA1, LA2, LA3, LA4, LA5, LA6, LA7, and combinations thereof.

18. The method of claim 16, wherein the at least one LDLR type A domain comprises at least one tandem domain selected from LA1-2, LA2-3, LA3-4, LA4-5, LA5-6, LA6-7, and combinations thereof.

19. The method of claim 15, wherein the HlyA inhibiting agent comprises a clathrin-mediated endocytosis (CME) inhibitor.

20. The method of claim 15, wherein the HlyA inhibiting agent comprises an anti-LDLR antibody.
